SEQUENCE LISTING (89 sequences)

SEQ ID NO: 1 - protein, 4 aa, Artificial
  Synthetic peptide
EVET

SEQ ID NO: 2 - protein, 15 aa, Artificial
  Synthetic peptide
MSLLTEVETHTRNGW

SEQ ID NO: 3 - protein, 9 aa, Artificial
  Synthetic peptide
PIRNEWGCR

SEQ ID NO: 4 - protein, 10 aa, Artificial
  Synthetic peptide
ETPIRNEWGC

SEQ ID NO: 5 - protein, 11 aa, Artificial
  Synthetic peptide
PIRNEWGCRCN

SEQ ID NO: 6 - protein, 9 aa, Artificial
  Synthetic peptide
LLTEVETPI

SEQ ID NO: 7 - protein, 9 aa, Artificial
  Synthetic peptide
SLLTEVETP

SEQ ID NO: 8 - protein, 10 aa, Artificial
  Synthetic peptide
SLLTEVETPI

SEQ ID NO: 9 - protein, 9 aa, Artificial
  Synthetic peptide
LTEVETPLT

SEQ ID NO: 10 - protein, 15 aa, Artificial
  Synthetic peptide
MSLLTEVETPIRNEW

SEQ ID NO: 11 - protein, 18 aa, Artificial
  Synthetic peptide
MSLLTEVETPIRNEWGCR

SEQ ID NO: 12 - protein, 15 aa, Artificial
  Synthetic peptide
MSLLTEVETLTKNGW

SEQ ID NO: 13 - protein, 15 aa, Artificial
  Synthetic peptide
MSLLTEVETLTRNGW

SEQ ID NO: 14 - protein, 9 aa, Artificial
  Synthetic peptide
LTEVETPIR

SEQ ID NO: 15 - protein, 10 aa, Artificial
  Synthetic peptide
LTEVETPIRN

SEQ ID NO: 16 - protein, 9 aa, Artificial
  Synthetic peptide
EVETPIRNE

SEQ ID NO: 17 - protein, 10 aa, Artificial
  Synthetic peptide
EVETPIRNEW

SEQ ID NO: 18 - protein, 11 aa, Artificial
  Synthetic peptide
LTEVETPIRNE

SEQ ID NO: 19 - protein, 15 aa, Artificial
  Synthetic peptide
LTEVETPIRNEWGCR

SEQ ID NO: 20 - protein, 8 aa, Artificial
  Synthetic peptide
EVETPIRN

SEQ ID NO: 21 - protein, 18 aa, Artificial
  Synthetic peptide
MSLLTEVETPTRNEWECR

SEQ ID NO: 22 - protein, 23 aa, Artificial
  Synthetic peptide
SLLTEVETPTRNEWECRCSDSSD

SEQ ID NO: 23 - protein, 23 aa, Artificial
  Synthetic peptide
SLLTEVETPIRNEWGCRCNDSSD

SEQ ID NO: 24 - protein, 9 aa, Artificial
  Synthetic peptide
VETPIRNEW

SEQ ID NO: 25 - protein, 11 aa, Artificial
  Synthetic peptide
SLLTEVETYVL

SEQ ID NO: 26 - protein, 11 aa, Artificial
  Synthetic peptide
SLLTEVETYVP

SEQ ID NO: 27 - protein, 9 aa, Artificial
  Synthetic peptide
LLTEVETYV

SEQ ID NO: 28 - protein, 8 aa, Artificial
  Synthetic peptide
SIVPSGPL

SEQ ID NO: 29 - protein, 15 aa, Artificial
  Synthetic peptide
SGPLKAEIAQRLEDV

SEQ ID NO: 30 - protein, 12 aa, Artificial
  Synthetic peptide
GPLKAEIAQRLE

SEQ ID NO: 31 - protein, 9 aa, Artificial
  Synthetic peptide
RLEDVFAGK

SEQ ID NO: 32 - protein, 11 aa, Artificial
  Synthetic peptide
ALMEWLKTRPI

SEQ ID NO: 33 - protein, 10 aa, Artificial
  Synthetic peptide
PILSPLTKGI

SEQ ID NO: 34 - protein, 9 aa, Artificial
  Synthetic peptide
ILSPLTKGI

SEQ ID NO: 35 - protein, 19 aa, Artificial
  Synthetic peptide
LTKGILGFVFTLTVPSERG

SEQ ID NO: 36 - protein, 13 aa, Artificial
  Synthetic peptide
TKGILGFVFTLTV

SEQ ID NO: 37 - protein, 12 aa, Artificial
  Synthetic peptide
KGILGFVFTLTV

SEQ ID NO: 38 - protein, 9 aa, Artificial
  Synthetic peptide
GILGFVFTL

SEQ ID NO: 39 - protein, 9 aa, Artificial
  Synthetic peptide
LGFVFTLTV

SEQ ID NO: 40 - protein, 9 aa, Artificial
  Synthetic peptide
ILGFVFTLT

SEQ ID NO: 41 - protein, 8 aa, Artificial
  Synthetic peptide
ASCMGLIY

SEQ ID NO: 42 - protein, 9 aa, Artificial
  Synthetic peptide
RMGAVTTEV

SEQ ID NO: 43 - protein, 11 aa, Artificial
  Synthetic peptide
GLVCATCEQIA

SEQ ID NO: 44 - protein, 9 aa, Artificial
  Synthetic peptide
QMVATTNPL

SEQ ID NO: 45 - protein, 10 aa, Artificial
  Synthetic peptide
QMVATTNPLI

SEQ ID NO: 46 - protein, 10 aa, Artificial
  Synthetic peptide
RMVLASTTAK

SEQ ID NO: 47 - protein, 9 aa, Artificial
  Synthetic peptide
DLLENLQTY

SEQ ID NO: 48 - protein, 18 aa, Artificial
  Synthetic peptide
SKAYSNCYPYDVPDYASL

SEQ ID NO: 49 - protein, 18 aa, Artificial
  Synthetic peptide
SKAFSNCYPYDVPDYASL

SEQ ID NO: 50 - protein, 18 aa, Artificial
  Synthetic peptide
STAYSNCYPYDVPDYASL

SEQ ID NO: 51 - protein, 7 aa, Artificial
  Synthetic peptide
WTGVTQN

SEQ ID NO: 52 - protein, 10 aa, Artificial
  Synthetic peptide
WLTGKNGLYP

SEQ ID NO: 53 - protein, 10 aa, Artificial
  Synthetic peptide
WLTEKEGSYP

SEQ ID NO: 54 - protein, 19 aa, Artificial
  Synthetic peptide
PKYVKQNTLKLATGMRNVP

SEQ ID NO: 55 - protein, 11 aa, Artificial
  Synthetic peptide
GVKLESMGIYQ

SEQ ID NO: 56 - protein, 11 aa, Artificial
  Synthetic peptide
EISGVKLESMG

SEQ ID NO: 57 - protein, 10 aa, Artificial
  Synthetic peptide
NVKNLYEKVK

SEQ ID NO: 58 - protein, 18 aa, Artificial
  Synthetic peptide
KVKILPKDRWTQHTTTGG

SEQ ID NO: 59 - protein, 13 aa, Artificial
  Synthetic peptide
PKYVKQNTLKLAT

SEQ ID NO: 60 - protein, 9 aa, Artificial
  Synthetic peptide
KTGGPIYRR

SEQ ID NO: 61 - protein, 9 aa, Artificial
  Synthetic peptide
CTELKLSDY

SEQ ID NO: 62 - protein, 14 aa, Artificial
  Synthetic peptide
HPSAGKDPKKTGGP

SEQ ID NO: 63 - protein, 13 aa, Artificial
  Synthetic peptide
HPSAGKDPKKTGG

SEQ ID NO: 64 - protein, 24 aa, Artificial
  Synthetic peptide
FWRGENGRKTRSAYERMCNILKGK

SEQ ID NO: 65 - protein, 9 aa, Artificial
  Synthetic peptide
ILRGSVAHK

SEQ ID NO: 66 - protein, 9 aa, Artificial
  Synthetic peptide
KLLQNSQVY

SEQ ID NO: 67 - protein, 15 aa, Artificial
  Synthetic peptide
SAAFEDLRVLSFIRG

SEQ ID NO: 68 - protein, 16 aa, Artificial
  Synthetic peptide
SAAFEDLRVSSFIRGT

SEQ ID NO: 69 - protein, 16 aa, Artificial
  Synthetic peptide
SAAFEDLRVLSFIRGY

SEQ ID NO: 70 - protein, 14 aa, Artificial
  Synthetic peptide
ELRSRYWAIRTRSG

SEQ ID NO: 71 - protein, 9 aa, Artificial
  Synthetic peptide
ELRSRYWAI

SEQ ID NO: 72 - protein, 9 aa, Artificial
  Synthetic peptide
SRYWAIRTR

SEQ ID NO: 73 - protein, 10 aa, Artificial
  Synthetic peptide
YWAIRTRSGG

SEQ ID NO: 74 - protein, 9 aa, Artificial
  Synthetic peptide
SRYWAIRTR

SEQ ID NO: 75 - protein, 9 aa, Artificial
  Synthetic peptide
LPFDKPTIM

SEQ ID NO: 76 - protein, 9 aa, Artificial
  Synthetic peptide
VSDGGPNLY

SEQ ID NO: 77 - protein, 9 aa, Artificial
  Synthetic peptide
RRSFELKKL

SEQ ID NO: 78 - protein, 9 aa, Artificial
  Synthetic peptide
RRATAILRK

SEQ ID NO: 79 - protein, 9 aa, Artificial
  Synthetic peptide
RPIIRPATL

SEQ ID NO: 80 - protein, 9 aa, Artificial
  Synthetic peptide
ADRGLLRDI

SEQ ID NO: 81 - protein, 13 aa, Artificial
  Synthetic peptide
PYYTGEHAKAIGN

SEQ ID NO: 82 - protein, 19 aa, Artificial
  Synthetic peptide
PAKLLKERGFFGAIAGFLE

SEQ ID NO: 83 - DNA, 2199 bp, Artificial
  Synthetic polynucleotide
atgcatatgagatctccagctaaacttctgaaagaacgtggatttttcggtgcaatcgctggttttctggaggggtcgaaagcctacagtaactgttacccctacgatgtgcccgattatgccagcctgggtagcctccttacagaagttgaaacttatgtgctcggctggctgacagggaaaaacggcctttatcctgtgtggaccggcgtgacgcagaacggattctggcgtggcgaaaatggacgtaaaactcgcagtgcgtatgagcgcatgtgtaacatcctcaaaggtaaaggcccgaaatatgtgaaacagaatacattaaaattagccaccggcgcgagcgctgcctttgaagacctccgtgtgctcagttttatccgcggttatggggaactgcgttctcgctattgggcgatccgtacccggtcagggggtccaccggcgaagctgctgaaagaacgtgggttcttcggtgcgattgccggtttcttggaaggatcaaaagcgtattcgaactgctacccgtatgatgtgccagattacgccagcctgggctccctcttgacagaggtcgaaacctatgtactgggttggctgaccggtaagaacggtctgtatccggtttggactggtgtgacacaaaacggcttttggcggggggaaaacggccggaaaacccgcagcgcttacgagcgcatgtgcaacattctgaaaggcaaaggcccgaaatacgtgaagcagaatacgctcaaacttgccacgggcgcaagcgcagcctttgaagacctgcgggtcttgagctttatccgcggttacggggagctgcggtcgcgctactgggcgattcgtacgcgtagtggtggacctcccgcgaaacttctgaaagagcggggcttctttggagcgattgcgggcttcttggagggaagcaaagcctactctaattgttacccatacgatgtgcctgattatgcgagcctcggtagcttgctgacagaagtggaaacctacgttctcggctggctgacgggcaaaaatggtctctacccagtgtggaccggagttacccagaatgggttctggcgcggtgagaacggccgtaaaacacgttcagcgtacgagcggatgtgcaacatcttaaaaggcaaaggaccgaaatacgtcaagcagaatactctgaagttagccactggggcctcagccgcctttgaagaccttcgcgtcttgagttttatccggggttatggggaactgcggagccgctactgggctattcgtacgcggtcgggtggcccactcgagccggccaaattgctcaaagaacgtggtttcttcggagcgatcgcaggttttcttgaaggctctaaagcgtacagcaactgttatccatacgatgtgccggattacgccagtctgggttccctcctgaccgaggtggaaacgtatgtactaggatggctcacgggtaaaaatggtctctatcctgtgtggacgggcgtaacccagaacggcttttggcggggcgaaaacggccgcaaaacccgtagcgcatacgagcgtatgtgtaacatccttaaaggcaaaggtccaaaatacgttaagcagaataccctgaaactggctacgggcgccagtgcggccttcgaagatttacgggtgctgtccttcatccgcggctatggtgaactgcgctctcgttactgggcaatccgtacccgcagtggcggacctccggctaaactgttgaaagaacgcggcttctttggtgctatcgcaggttttctggaaggaagtaaagcatattcgaattgttatccctacgacgtgccggattatgcgtcgctcggttcgctgctgaccgaggtggaaacctacgttctaggctggttgacaggtaagaacgggctttacccggtatggaccggcgttacccagaacggtttttggcgcggtgaaaatggccgtaaaactcggtcagcatacgaacggatgtgcaatatcttgaaaggtaaaggaccgaaatacgttaaacagaacacgctgaaactggcaacaggcgccagcgcggcgtttgaggatttacgcgtcctgtcatttattcggggctacggcgaattacgtagtcgttattgggcgattcgtacccgcagcggagggctcgagtaataaaagctttctagacatatgatgcat

SEQ ID NO: 84 - protein, 723 aa, Artificial
  Synthetic polypeptide
MHMRSPAKLLKERGFFGAIAGFLEGSKAYSNCYPYDVPDYASLGSLLTEVETYVLGWLTGKNGLYPVWTGVTQNGFWRGENGRKTRSAYERMCNILKGKGPKYVKQNTLKLATGASAAFEDLRVLSFIRGYGELRSRYWAIRTRSGGPPAKLLKERGFFGAIAGFLEGSKAYSNCYPYDVPDYASLGSLLTEVETYVLGWLTGKNGLYPVWTGVTQNGFWRGENGRKTRSAYERMCNILKGKGPKYVKQNTLKLATGASAAFEDLRVLSFIRGYGELRSRYWAIRTRSGGPPAKLLKERGFFGAIAGFLEGSKAYSNCYPYDVPDYASLGSLLTEVETYVLGWLTGKNGLYPVWTGVTQNGFWRGENGRKTRSAYERMCNILKGKGPKYVKQNTLKLATGASAAFEDLRVLSFIRGYGELRSRYWAIRTRSGGPLEPAKLLKERGFFGAIAGFLEGSKAYSNCYPYDVPDYASLGSLLTEVETYVLGWLTGKNGLYPVWTGVTQNGFWRGENGRKTRSAYERMCNILKGKGPKYVKQNTLKLATGASAAFEDLRVLSFIRGYGELRSRYWAIRTRSGGPPAKLLKERGFFGAIAGFLEGSKAYSNCYPYDVPDYASLGSLLTEVETYVLGWLTGKNGLYPVWTGVTQNGFWRGENGRKTRSAYERMCNILKGKGPKYVKQNTLKLATGASAAFEDLRVLSFIRGYGELRSRYWAIRTRSGGLE

SEQ ID NO: 85 - DNA, 1299 bp, Artificial
  Synthetic polynucleotide
atgcatatgagatctccagctaaacttctgaaagaacgtggatttttcggtgcaatcgctggttttctggagccaccggcgaagctgctgaaagaacgtgggttcttcggtgcgattgccggtttcttggaacctcccgcgaaacttctgaaagagcggggcttctttggagcgattgcgggcttcttggagccatcgaaagcctacagtaactgttacccctacgatgtgcccgattatgccagcctgccttcaaaagcgtattcgaactgctacccgtatgatgtgccagattacgccagcctgccaagcaaagcctactctaattgttacccatacgatgtgcctgattatgcgagcctccctagcctccttacagaagttgaaacttatgtgctcagcttgctgacagaagtggaaacctacgttctcagcttgctgacagaagtggaaacctacgttctctggctgacagggaaaaacggcctttatccttggctgaccggtaagaacggtctgtatccgtggctgacgggcaaaaatggtctctacccatggaccggcgtgacgcagaacccttggactggtgtgacacaaaacccatggaccggagttacccagaatcctttctggcgtggcgaaaatggacgtaaaactcgcagtgcgtatgagcgcatgtgtaacatcctcaaaggtaaacccttttggcggggggaaaacggccggaaaacccgcagcgcttacgagcgcatgtgcaacattctgaaaggcaaaccattctggcgcggtgagaacggccgtaaaacacgttcagcgtacgagcggatgtgcaacatcttaaaaggcaaacctccgaaatacgtgaagcagaatacgctcaaacttgccacgccaccgaaatacgtcaagcagaatactctgaagttagccactccgccgaaatacgtcaagcagaatactctgaagttagccactccttcagccgcctttgaagaccttcgcgtcttgagttttatccggggttatccaagcgcagcctttgaagacctgcgggtcttgagctttatccgcggttacccttcagccgcctttgaagaccttcgcgtcttgagttttatccggggttatccagaactgcgttctcgctattgggcgatccgtacccggtcagggccggagctgcggtcgcgctactgggcgattcgtacgcgtagtggtccagaactgcggagccgctactgggctattcgtacgcggtcgggttaataactcgagaggctttctagacatatgatgcat

SEQ ID NO: 86 - protein, 421 aa, Artificial
  Synthetic polypeptide
MHMRSPAKLLKERGFFGAIAGFLEPPAKLLKERGFFGAIAGFLEPPAKLLKERGFFGAIAGFLEPSKAYSNCYPYDVPDYASLPSKAYSNCYPYDVPDYASLPSKAYSNCYPYDVPDYASLPSLLTEVETYVLSLLTEVETYVLSLLTEVETYVLWLTGKNGLYPWLTGKNGLYPWLTGKNGLYPWTGVTQNPWTGVTQNPWTGVTQNPFWRGENGRKTRSAYERMCNILKGKPFWRGENGRKTRSAYERMCNILKGKPFWRGENGRKTRSAYERMCNILKGKPPKYVKQNTLKLATPPKYVKQNTLKLATPPKYVKQNTLKLATPSAAFEDLRVLSFIRGYPSAAFEDLRVLSFIRGYPSAAFEDLRVLSFIRGYPELRSRYWAIRTRSGPELRSRYWAIRTRSGPELRSRYWAIRTRSG

SEQ ID NO: 87 - DNA, 1335 bp, Artificial
  Synthetic polynucleotide
atgagatctccggcgaaactgctgaaagaacgtggcttttttggcgcgattgcgggctttctggaaggcagcaaagcgtatagcaactgctatccgtatgatgtgccggattacgcgagtctgggctctctgctgaccgaagtggaaacctatgtgctgggctggctgaccggcaaaaacggcctgtatccggtgtggaccggcgtgacccagaacggcttttggcgtggcgaaaacggccgtaaaacccgtagcgcgtatgaacgtatgtgcaacatcctgaaaggcaaaggcccgaaatatgtgaaacagaacaccctgaaactggccaccggtgcgagcgcggcgtttgaggacctgcgtgttctgagctttattcgtggctatggcgaactgcgtagccgttattgggcgattcgtacccgtagcggtggtccgccggccaaactgctgaaagaacgcggtttcttcggtgcgatcgccggttttctggaaggtagcaaagcctactctaattgttacccgtacgatgttccggattacgccagcctgggtagcctgctgaccgaagttgaaacctacgttctgggttggctgaccggtaaaaatggtctgtacccggtttggaccggtgttacccagaatggtttctggcgcggtgaaaatggtcgcaaaacccgcagcgcctacgaacgcatgtgtaatattctgaaaggtaaaggtccgaaatacgttaaacagaataccctgaaactggccaccggcgccagcgccgccttcgaggacctgcgcgttctgagcttcatccgcggttacggtgaactgcgcagccgctactgggccatccgcacccgcagcggtggtccgccggcgaaactgctgaaagaacgcggtttttttggtgccattgcgggttttctggaaggtagcaaagcctattctaactgctatccgtacgatgttccggattatgcgagcctgggtagcctgctgaccgaagtggaaacctatgttctgggttggctgaccggcaaaaacggtctgtatccggtttggaccggtgtgacccagaacggtttttggcgcggtgaaaacggccgtaaaacccgcagcgcctatgaacgcatgtgcaacattctgaaaggcaaaggtccgaaatacgtgaaacagaacaccctgaaactggccaccggcgcgagcgcggcctttgaggacctgcgcgttctgagctttattcgcggctatggtgaactgcgcagccgctattgggcgattcgtacccgcagcggcggctaataactcgagaagctttctagacatatgatgcatgagctc

SEQ ID NO: 88 - protein, 431 aa, Artificial
  Synthetic polypeptide
MRSPAKLLKERGFFGAIAGFLEGSKAYSNCYPYDVPDYASLGSLLTEVETYVLGWLTGKNGLYPVWTGVTQNGFWRGENGRKTRSAYERMCNILKGKGPKYVKQNTLKLATGASAAFEDLRVLSFIRGYGELRSRYWAIRTRSGGPPAKLLKERGFFGAIAGFLEGSKAYSNCYPYDVPDYASLGSLLTEVETYVLGWLTGKNGLYPVWTGVTQNGFWRGENGRKTRSAYERMCNILKGKGPKYVKQNTLKLATGASAAFEDLRVLSFIRGYGELRSRYWAIRTRSGGPPAKLLKERGFFGAIAGFLEGSKAYSNCYPYDVPDYASLGSLLTEVETYVLGWLTGKNGLYPVWTGVTQNGFWRGENGRKTRSAYERMCNILKGKGPKYVKQNTLKLATGASAAFEDLRVLSFIRGYGELRSRYWAIRTRSGG

SEQ ID NO: 89 - protein, 13 aa, Artificial
  Synthetic peptide
PRYVKQNTLKLAT

(19) United States
(12) Patent Application Publication
(10) Pub. No.: US 2020/0000910 A1
(43) Pub. Date: Jan. 2, 2020

(54) COMPOSITIONS OF MULTIMERIC-MULTIEPITOPE INFLUENZA POLYPEPTIDES AND THEIR PRODUCTION

(71) Applicant: BIONDVAX PHARMACEUTICALS LTD., NESS ZIONA (IL)

(72) Inventors: Ram URITSKI, Rehovot (IL); Barry COHEN, Rehovot (IL); Zohar GADRI, Rehovot (IL); Shimon HASSIN, Tel Aviv (IL)

(73) Assignee: BIONDVAX PHARMACEUTICALS LTD., NESS ZIONA (IL)

(21) Appl. No.: 16/551,991

(22) Filed: Aug. 27, 2019

Related U.S. Application Data
(63) Continuation-in-part of application No. 15/300,529, filed on Sep. 29, 2016, now Pat. No. 10,426,828.
(60) Provisional application No. 61/974,449, filed on Apr. 3, 2014.

(51) Int. Cl. A61K 39/145; A61K 9/16; C12N 7/00

(52) U.S. Cl. CPC A61K 39/145; A61K 2039/543; C12N 7/00; A61K 9/16

(57) ABSTRACT

The present invention relates to pharmaceutical comprising multimeric-multiepitope influenza polypeptides, to processes for their production and to their use as immunizing subjects against influenza. In particular, the invention relates to stable aqueous microparticulate suspensions and solid compositions comprising a multimeric multiepitope polypeptide.

### CROSS REFERENCE TO RELATED APPLICATIONS

This application is a Continuation-in-Part Application of U.S. application Ser. No. 15/300,529, filed on Sep. 29, 2016; which is a National Phase Application of PCT International Application No. PCT/IL2015/050354, filed on Apr. 1, 2015, which claims priority to U.S. Application No. 61/974,449, filed on Apr. 3, 2014, which are hereby incorporated by reference in their entirety.

### SEQUENCE LISTING

The Sequence Listing submitted herewith as an ASCII text file (2019-08-27_Sequence_Listing.txt, created on Aug. 27, 2019, 37971 bytes) via EFS-Web is hereby incorporated by reference.

### FIELD OF THE INVENTION

The present invention relates to pharmaceutical compositions of multimeric multi-epitope peptide-based influenza vaccines and to methods of their preparation. Specifically, the present invention provides microparticulate suspensions and dry compositions comprising a multimeric multi-epitope polypeptide and a guanidinium-containing amino acid, their preparation and their use for protecting subjects against influenza.

### BACKGROUND OF THE INVENTION

Influenza is a highly infectious disease caused by rapidly mutating influenza viruses. It is easily transmitted and spreads around the world in seasonal epidemics, infecting 5-20% of the total population annually. According to the World Health Organization (WHO), 250,000-500,000 people die annually of influenza-related causes during seasonal outbreaks. In the USA alone, more than 200,000 people are hospitalized with seasonal influenza in a typical year. Influenza infection may be mild, moderate or severe, ranging from asymptomatic through mild upper respiratory infection and tracheobronchitis to a severe, occasionally lethal, viral pneumonia. The infection is associated with pulmonary and cardiovascular complications leading to high morbidity and mortality rates, affecting mainly at-risk populations such as toddlers, elderly and individuals with chronic medical conditions.

Of the three types of influenza viruses, Influenza A and Influenza B are responsible for approximately 80% and 20% of influenza disease in humans, respectively, while influenza C viruses do not infect humans. Influenza A viruses are characterized by many sub-strains and by species specificity and are considered to be the major cause of widespread seasonal epidemics and of pandemics, due to the frequent antigenic drifts and shifts of the Hemagglutinin (HA) and Neuraminidase (NA) surface proteins. Following antigenic changes, infection via virus strains which are unrecognized by the immune system may result in a reduced immune response by the infected individual, where more significant changes will yield less effective stimulation of the body's immune defenses. Antigenic drifts or shifts can trigger respective influenza epidemics or pandemics, as experienced with the recent Avian and Swine Flu pandemic strains.

To date, commercially available influenza vaccines contain influenza A and B antigens that are annually selected according to predictions of the strains to be most prevalent during the peak influenza season. However, due to the mismatch between the strains included in the vaccine and those actually circulating, these strain-specific vaccines often have relatively poor clinical efficacy. In addition, such immunization methods require preparation of new vaccine formulations on an annual basis. Thus, a vaccine recognizing multiple virus strains would be more cost effective and would further increase patient compliance and enhance global health prospects.

Commercial influenza vaccine compositions currently in use are aqueous solutions comprising typically phosphate, sodium, potassium and/or calcium buffers with additions of Triton, Tween, α-tocopheryl hydrogen succinate and/or other additives or excipients. FLUMIST™ (MedImmune), a live attenuated vaccine for influenza that presents the surface antigens of seasonal flu, is supplied as a solution comprising about 0.05 M arginine, 0.188 mg monosodium glutamate, 2 mg hydrolyzed porcine gelatin, 13.68 mg sucrose, 2.26 mg dibasic potassium phosphate, and 0.96 mg monobasic potassium phosphate.

PCT International Publication WO 2009/016639 to some of the inventors of the present invention discloses influenza multi-epitope polypeptides and vaccines comprising a plurality of influenza virus peptide epitopes wherein each epitope is present at least twice in a single polypeptide.

The Multimeric-001 (M-001) vaccine consists of nine conserved linear epitopes arranged as three repetitions of each and prepared as a single, recombinant polypeptide expressed in Escherichia coli (E. coli). These epitopes are common to the vast majority of influenza virus strains, regardless of their antigenic drifts and shifts. Consequently, M-001 is expected to provide immunity-based protection against future virus strains as well. Multimeric-001 vaccination leads to efficient cross-strain recognition and protection despite variations in the outer proteins of each strain.

The significant results obtained with various animal models and the safety parameters observed in the repeated toxicology study have paved the way toward, and provided the foundation for clinical trials in humans. Phase I/II and Phase II clinical trials assessing the safety and immunogenicity of M-001 in adult and elderly volunteers were completed (Atsmon et al., 2014, Vaccine 32, 5816-5823). Doses of 125-500 μg adjuvanted or non-adjuvanted vaccine, in PBS, proved safe and well tolerated. Potential Multimeric-001 vaccine-related toxicity was evaluated in GLP toxicology studies. Both M-001 vaccine formulations (adjuvanted and non-adjuvanted) repeatedly IM administered at the maximal human dose, proved to be safe. A Pivotal Phase 3 clinical trial aims to assess safety and clinical efficacy of M-001 as a standalone vaccine by reducing flu illness and its severity, the trial is currently ongoing and expected to enroll about 12,000 subjects in total (NCT03450915).

WO 2012/114323 to some of the inventors of the present invention provides a method of improving the protective effect of a seasonal or pandemic influenza vaccine by administering to a subject, prior to or together with the influenza vaccine, a multimeric influenza polypeptide comprising multiple copies of plurality of influenza virus peptide epitopes.

Formation of inclusion bodies (IBs) frequently occurs when heterologous proteins are expressed in E. coli, and recovery of the active recombinant protein often requires refolding into its active structure. Wingfield, P. T. (Current Protocols in Protein Science. 2003, 30:6.1.1-6.1.37) reviewed the purification of recombinant proteins produced in E. coli.

Arginine is used for refolding and purification of proteins obtained from IBs and appears to be effective for a variety of proteins differing in chemical and physical properties. The role of arginine in protein refolding, solubilization and purification was reviewed by Tsumoto et al., (Biotechnol Prog. 2004 September-October; 20(5):1301-8; Schneider et al., (J Phys Chem B. 2011 Jun. 9; 115(22):7447-58)).

An anti-aggregation effect and stabilizing effect of arginine was previously described. For example, Lyutova et al., (Biotechnol Prog. 2007 November-December; 23(6):1411-6) studied the effect of low concentrations of arginine (1-10 mM) on protein aggregation, when protein aggregation is induced by transition from a folded state by heating or by addition of dithiothreitol (DTT).

Low-molecular-weight additives, such as L-arginine, have been suggested to enhance renaturation yields by inhibiting intermolecular hydrophobic interactions that lead to precipitation. Ho et al., (Protein Sci. 2003, 12, 708-716) demonstrated that L-arginine suppresses aggregation by increasing protein solubility.

Production of a protein in the form of a pharmaceutical suspension is generally favored as a result of one or more of the following reasons: the solubility of the polypeptide; the stability of the polypeptide; controlling or altering the release profile of the polypeptide.

Suspensions of protein micro- or nano-particles may be produced by a number of methods. Such particles may be initially produced as larger particles, followed by size reduction procedure by physical or chemical means. Some other approaches include crystallization, lyophilization, spray-drying and supercritical fluid particle formation or desolvation.

WO 2009/015736 discloses a process of production of a purified recombinant GDF-5 related protein comprising treatment of the inclusion bodies with a denaturing solubilization buffer comprising L-arginine.

WO 2012/054679 discloses methods for purifying a recombinant protein from a mixture comprising the recombinant protein and inclusion bodies, the method comprising: a) solubilizing the mixture comprising the recombinant protein with associated inclusion bodies with a solubilization buffer comprising ethanolamine, arginine, EDTA, urea and DTE.

US 2003/0199441 relates to a method to produce renatured procollagen propeptides wherein inclusion bodies produced in E. coli are dissolved in a 0.5 to 8 M denaturing buffer which is then added dropwise into a limited dilution buffer that is buffered around neutral pH and contains L-arginine in a final concentration between 200 to 1,000 nM and a disulfide bridges-reducing coupled redox system, and then the buffer mixture is dialyzed against a physiological buffer that contains L-arginine at a final concentration of 50 to 200 nM and a disulfide bridges-reducing coupled redox system and later against a physiological buffer that contains a disulfide bridges-reducing coupled redox system and finally against a physiological buffer.

US 2004/0137588 describes a method of purifying polypeptides from a biological sample subjecting the polypeptide to refolding conditions in the presence of arginine.

It would be advantageous to have stable suspensions of microparticulate polypeptides having a uniform size distribution, and solid compositions thereof, for use in improved multimeric-multiepitope influenza vaccines. Efficient production processes of such compositions are also required.

### SUMMARY OF THE INVENTION

The present invention provides improved influenza vaccine suspensions comprising at least one multimeric influenza polypeptide comprising multiple copies of a plurality of influenza virus peptide epitopes, herein denoted multimeric-multiepitope polypeptide, and a guanidinium-containing amino acid or a derivative thereof. Methods of producing such suspension compositions are also provided as well as solid compositions produced from these suspensions.

The present invention is based in part on the finding that controlled generation of protein aggregates during removal of chaotropic and reducing agents from the multimeric-multiepitope polypeptide solution, together with addition of the amino acid arginine, results in stable and uniform microparticulate suspensions of the multimeric-multiepitope polypeptide at a desired protein concentration and pH which is close to a physiological pH, overcoming the need for high pressure homogenization in order to convert large amorphous aggregates into injectable formulations.

According to some embodiments, the present invention provides a pharmaceutical composition in the form of an aqueous suspension of microparticles, said composition comprising at least one multimeric-multiepitope polypeptide comprising multiple copies of plurality of influenza virus peptide epitopes, a guanidinium-containing amino acid or a derivative thereof, and a pharmaceutically-acceptable diluent, excipient or carrier, wherein the aqueous suspension comprises aggregates of microparticles of said multimeric-multiepitope polypeptide having uniform aggregate size distribution with 95% of the aggregates in the suspension having a size range distribution selected from the group consisting of: 0.5-5 μm, 0.6-6 μm, 0.7-7 μm, 0.8-8 μm, 0.9-9 μm, 1-10 μm, 2-20 μm, 3-30 μm, 4-40 μm and 5-50 μm.

According to some embodiments, the guanidinium-containing amino acid is an arginine (Arg).

According to some embodiments, the composition comprises a buffering agent at a concentration of 1-50 mM that maintains a pH within the range of 5.0 to 7.6.

According to some embodiments, the pharmaceutical composition comprises 1-10 mg/ml of multimeric-multiepitope influenza polypeptide, 0.1-0.5 M of L-arginine and 10-50 mM citrate buffer, and having a pH in the range of 4 to 7.

According to some embodiments, the multimeric-multiepitope polypeptide comprises 3-5 repeats of 4-9 peptide epitopes each epitope is selected from the group consisting of: HA 354-372 (E1, SEQ ID NO: 82), HA 91-108 (E2, SEQ ID NO: 48), M1 2-12 (E3, SEQ ID NO: 25), HA 150-159 (E4, SEQ ID NO: 52), HA 143-149 (E5, SEQ ID NO: 51), NP 206-229 (E6, SEQ ID NO: 64), HA 307-319 (E7, SEQ ID NO: 59 or 89), NP 335-350 (E8, SEQ ID NO: 69), and NP 380-393 (E9, SEQ ID NO: 70).

According to some embodiments, the multimeric-multiepitope influenza polypeptide is M-001 having an amino acid sequence set forth in SEQ ID NO: 86.

According to some embodiments, the pharmaceutical composition is in a form suitable for administration in a route selected from injectable, oral, intranasal, sublingual, topical and rectal.

According to some embodiments, the aqueous suspension of microparticles is produced by a process comprising the steps of:
i. providing at least one multimeric multiepitope influenza polypeptide at a concentration of 1.5-5 mg/ml in an aqueous solution comprising a chaotropic agent, a detergent, a reducing agent, and a buffering agent providing a pH in the range of 7-10;
ii. inducing of controlled aggregation by gradual removal of the chaotropic and reducing agents; and
iii. adding a guanidinium-containing compound to achieve a stable microparticulate suspension having a uniform aggregate size distribution, with 95% of the aggregate sizes falling into a size range of one order of magnitude.

According to some embodiments, the present invention further provides a process of producing a recombinant multimeric-multiepitope polypeptide composition, the process comprises the steps:
i. providing at least one multimeric multiepitope influenza polypeptide in an aqueous solution comprising a chaotropic agent, a detergent, a reducing agent, and a buffering agent, providing a pH in the range of 7-11;
ii. inducing of controlled aggregation by gradual removal of the chaotropic and reducing agents; and
iii. adding a guanidinium-containing amino acid or a derivative thereof to achieve a stable microparticulate suspension having a uniform aggregate size distribution, with 95% of the aggregate in the suspension having a size range distribution selected from the group consisting of: 0.5-5 μm, 0.6-6 μm, 0.7-7 μm, 0.8-8 μm, 0.9-9 μm, 1-10 μm, 2-20 μm, 3-30 μm, 4-40 μm and 5-50 μm,
wherein the at least one multimeric-multiepitope polypeptide comprises multiple copies of plurality of influenza virus peptide epitopes.

According to some embodiments, the process further comprises the steps of:
iv. freezing the stable microparticulate suspension of step (iii) at a temperature in the range of −10° C. to −50° C.; and
v. reducing the pressure of the frozen composition of step (iv) for a period of time, so as to sublime at least 95% of the water contained therein, thereby providing a solid composition of the multimeric multiepitope influenza polypeptide.

According to some embodiments, step (iv) comprises freezing the stable microparticulate suspension of step (iii) at a temperature in the range of −15° C. to −25° C. for 12 hours to 36 hours, and reducing said temperature to a temperature in the range of −30° C. to −50° C. for further 12 hours to 36 hours.

According to some embodiments, step (v) comprises reducing the pressure of the frozen composition of step (iv) for a period of 24 hours to 72 hours, so as to sublime at least 98% of the water contained therein, thereby providing the solid composition of the multimeric multiepitope influenza polypeptide.

According to some embodiments, step (i) comprises providing at least one multimeric multiepitope influenza polypeptide at a concentration of 1.5-5 mg/ml in an aqueous solution comprising a chaotropic agent, a detergent, a reducing agent, and a buffering agent providing a pH in the range of 7-10.

According to some embodiments, the chaotropic agent is 5-8 M urea and 1-4 M thiourea, the detergent is 0.5-4% CHAPS (3-[(3-cholamidopropyl) dimethylammonio]-1-propanesulfonate) and the buffering agent is 5-100 mM glycine, and the guanidinium-containing amino acid is an arginine (Arg).

According to some embodiments, the present invention further provides a pharmaceutical composition of multimeric multiepitope influenza polypeptide, comprising a stable microparticulate suspension prepared according to the process described herein, and a pharmaceutically-acceptable diluent, excipient or carrier.

According to some embodiments, the present invention further provides a solid pharmaceutical composition comprising a solid composition of a multimeric multiepitope influenza polypeptide prepared according to the process of disclosed herein, and a pharmaceutically-acceptable excipient or carrier.

According to some embodiments, the pharmaceutical composition is in the form of a powder.

According to some embodiments, the present invention further provides a packaged pharmaceutical composition comprised of a packaging material; and the solid pharmaceutical composition disclosed herein.

According to some embodiments, there is provided a method of inducing an immune response and conferring protection against influenza in a subject, comprising administering to the subject a pharmaceutical composition according to the present invention.

According to some embodiments, the administration is intranasal.

According to another aspect, the present invention provides a pharmaceutical composition in the form of an aqueous suspension, said pharmaceutical composition comprising at least one multimeric-multiepitope polypeptide, a guanidinium-containing amino acid or a derivative thereof, and a pharmaceutically-acceptable diluent, excipient or carrier.

According to some embodiments, the pharmaceutical composition is in the form of a vaccine for immunization of a subject against influenza.

According to some embodiments, the guanidinium-containing amino acid is an arginine (Arg).

According to other embodiments, the guanidinium-containing amino acid is an arginine derivative.

Any salt or free acid of L- or D-arginine, or of a derivative thereof, or mixture thereof, can be used according to the present invention.

According to some embodiments, the arginine is L-arginine (L-Arg).

According to some embodiments, the Arg compound is selected from the group consisting of: Arg-hydrochloride, Arg-sulfate, Arg-phosphate, Arg-citrate, Arg-acetate and Arg free acid. Each possibility represents a separate embodiment of the present invention.

According to some embodiments the composition comprises L-Arg hydrochloride (L-Arg HCl).

According to some embodiments, the pharmaceutical composition comprises a guanidinium-containing amino acid or a derivative thereof in a concentration of 0.1-2.0 M.

According to some embodiments, the pharmaceutical composition comprises L-arginine in a concentration of 0.15-0.45 M. According to other embodiments, the arginine concentration in the pharmaceutical composition is 0.15-0.30 M.

According to some embodiments, the pharmaceutical composition comprises an additional buffering agent having a concentration of 1-50 mM.

According to some embodiment, the buffering agent is selected from the group consisting of: citrate buffer, tris hydroxymethylaminomethane (Tris) buffer, HEPES (4-(2-hydroxyethyl)-1-piperazineethanesulfonate) buffer, lysine buffer, glycine buffer, MOPS (3-(N-morpholino)propanesulfonate) buffer, imidazole buffer and MES (2-(N-morpholino)ethanesulfonate) buffer. Each possibility represents a separate embodiment of the present invention.

According to specific embodiments, the composition comprises a buffer selected from the group consisting of: citrate buffer, lysine buffer and glycine buffer. Each possibility represents a separate embodiment of the present invention.

According to a particular embodiment, the composition comprises a citrate buffer.

According to some embodiments, the pH of the pharmaceutical composition is within the range of pH 5.0 to 7.6. According to particular embodiments, the pH of the pharmaceutical composition is within the range of pH 5.5 to 7.0. According to other particular embodiments, the buffering agent maintains a pH within the range of 5.7-6.5.

According to certain specific embodiments, a pharmaceutical composition is provided comprising 1-5 mg/ml of multimeric-multiepitope influenza polypeptide, 0.1-0.5 M of L-arginine and 10-50 mM citrate buffer, having a pH in the range of 5.0 to 7.0.

According to a specific embodiment, the pharmaceutical composition comprises 1-4 mg/ml of multimeric-multiepitope influenza polypeptide, 0.1-0.3 M L-arginine and 10-30 mM citrate buffer, having a pH in the range of 5.5 to 6.5.

According to a specific embodiment, the pharmaceutical composition comprises about 2.5 mg/ml of multimeric-multiepitope influenza polypeptide, about 0.2 M arginine, about 20 mM citrate buffer, and a pH of about 6.

According to some embodiments the pharmaceutical composition is a suspension comprising insoluble aggregates having a size distribution within the range of 0.5-50 μm wherein 95% of the aggregate sizes falling into a range of one order of magnitude.

According to some embodiments 95% of the aggregates in the suspension have a size distribution selected from the group consisting of: 0.5-5 μm, 1-10 μm, 3-30 μm and 5-50 μm. Each possibility represents a separate embodiment of the present invention.

According to some embodiments the multimeric-multiepitope influenza polypeptide is produced recombinantly.

According to some specific embodiments, the multimeric-multiepitope influenza polypeptide is denoted M-001 having an amino acid sequence as set forth in SEQ ID NO: 86.

According to another aspect, the present invention provides a process of producing a purified recombinant multimeric-multiepitope polypeptide suspension, comprising the steps:
i. solubilizing inclusion bodies containing at least one multimeric-multiepitope polypeptide in a solution comprising a chaotropic agent, a buffering agent, and a reducing agent, having a pH in a range of 7 to 11; and
ii. inducing controlled aggregation by gradual removal of the chaotropic and reducing agents consequently forming insoluble aggregate suspension;
iii. addition of arginine to achieve a stable and uniform suspension having a uniform aggregate size distribution, with 95% of the aggregate sizes falling into a size range of one order of magnitude.

According to specific embodiments the chaotropic agent comprises 5-8 M urea and 1-4 M thiourea.

According to particular embodiments, gradual removal of the chaotropic and reducing agents in step (ii) is performed by ultrafiltration.

According to some embodiments, the CHAPS in step (i) is present at a concentration of 0.5-2%.

According to some embodiments, the process comprises the steps:
i. providing E. coli cells expressing at least one multimeric-multiepitope influenza polypeptide;
ii. performing lysis and bacterial cell disruption to provide inclusion bodies comprising multimeric-multiepitope influenza polypeptide;
iii. solubilizing the inclusion bodies in a solution comprising 5-8M urea, 1-4 M thiourea, 0.5-4% CHAPS (3-[(3-cholamidopropyl)dimethylammonio]-1-propanesulfonate), a buffering agent, and a reducing agent, at a pH in the range of 7 toll;
iv. inducing controlled aggregation by gradual removal of the chaotropic and reducing agents consequently forming insoluble aggregate suspension;
v. addition of arginine to achieve a stable and uniform suspension having a uniform aggregate size distribution, with 95% of the aggregates falling into a size range of one order of magnitude.

According to some embodiments, gradual removal of chaotropic and reducing agents is performed by ultrafiltration using a procedure known in the art.

According to some embodiments, at least one chromatography separation step is performed between steps (iii) and (iv). According to some embodiments, the at least one chromatography separation step is selected from the group consisting of ion exchange chromatography and hydrophobic interaction chromatography.

According to some embodiments, step (v) is performed prior or during to aggregate formation of step (iv).

According to yet other embodiments, step (v) is performed following aggregate formation of step (iv).

According to some embodiments, the process comprises:
i. providing E. coli cells expressing at least one multimeric-multiepitope influenza polypeptide;
ii. performing lysis, bacterial cell disruption and centrifugation of the E. coli cells to provide inclusion bodies comprising multimeric-multiepitope influenza polypeptide;
iii. performing at least one wash of the inclusion bodies;
iv. solubilizing the inclusion bodies in a solution comprising 5-8M urea, 1-4 M thiourea, 0.5-4% CHAPS (3-[(3-cholamidopropyl)dimethylammonio]-1-propanesulfonate), a reducing agent and a buffering agent providing a pH in the range of 7-10;
v. subjecting the solution to at least one chromatography step;
vi. inducing of controlled aggregation by gradual removal of the chaotropic and reducing agents, thereby forming insoluble aggregates;
vii. addition of guanidinium-containing compound, and
viii. formulating the composition of (vii) to achieve a final suspension of about 0.1-0.4 M guanidinium-containing amino acid and 10-40 mM citrate buffer, at a pH in the range of 4 to 8.

According to other embodiments the addition of guanidinium-containing compound of step (vii) is performed gradually during induction of controlled aggregation.

According to some embodiments, lysis is performed in a buffer comprising Tris buffer, EDTA, and 0.1-0.5 mg lysozyme per 1 g of cell paste at a pH in the range of 7 to 9.

According to some embodiments, inducing of controlled aggregation of step (vi) is performed by ultrafiltration.

According to some embodiments, ultrafiltration comprises the steps of:
i. Buffer exchange I with 10-80 mM MES buffer pH 4-6.5;
ii. Buffer exchange II with 20-80 mM citrate buffer, 0.1-1 M arginine, pH 4-6;
iii. Buffer exchange III with 10-50 mM citrate buffer, 0.1-0.5 M arginine, pH 4.0 to 7.5.

According to other embodiments, ultrafiltration comprises the steps of:
i. Buffer exchange I with 10-80 mM MES buffer pH 4.0 to 6.5;
ii. Buffer exchange II with 20-80 mM citrate buffer pH 4 to 6;
iii. Dilution with arginine containing solution to 0.1-1 M arginine concentration.

According to some embodiments, the polypeptide concentration in the final composition is 1-10 mg/ml.

According to some embodiments, the at least one multimeric-multiepitope influenza polypeptide sequence is set forth in SEQ ID NO. 86.

According to some embodiments, the at least one multimeric-multiepitope influenza polypeptide is encoded by a polynucleotide sequence set forth in SEQ ID NO. 85.

According to some specific embodiments the process comprises the steps:
i. solubilizing inclusion bodies comprising recombinantly-produced multimeric-multiepitope influenza polypeptide in a solution comprising 6 M urea, 2 M thiourea, 1% CHAPS, 50 mM β-mercaptoethanol, 50 mM glycine, having a pH of about 9.5;
ii. inducing aggregation by gradual removal of the chaotropic and reducing agents (urea, thiourea and β-mercaptoethanol), thereby forming suspension of insoluble aggregates;
iii. subjecting the suspension to concentration and buffer-exchange steps by ultrafiltration comprising gradually addition of 0.5 M arginine buffer; and
iv. subjecting the suspension to buffer exchange to achieve a final composition comprising about 2.5 mg/ml of the polypeptide, about 0.2 M arginine and about 20 mM citrate buffer, having a pH about 6.

According to yet another aspect, the present invention provides a process of producing an essentially stable aqueous suspension of a recombinant multimeric-multiepitope polypeptide, comprising the steps of:
i. solubilizing inclusion bodies comprising recombinantly-produced multimeric-multiepitope influenza polypeptide in a solution comprising 6 M urea, 2 M thiourea, 1% CHAPS, 50 mM β-mercaptoethanol, 5-50 mM glycine, having a pH in the range of 7 to 10;
ii. inducing aggregation by gradual removal of the chaotropic and reducing agents, thereby forming suspension of insoluble aggregates;
iii. subjecting the suspension to concentration and buffer-exchange steps by ultrafiltration comprising gradually addition of an arginine buffer or a citrate buffer; and
iv. subjecting the suspension to buffer exchange to achieve a final stable and uniform suspension comprising about 1-5 mg/ml of the polypeptide, about 0.1-0.5 M arginine and about 10-50 mM citrate buffer, having a pH in the range of 4 to 7.

According to some embodiments, the process comprises the steps:
i. solubilizing inclusion bodies comprising recombinantly-produced multimeric-multiepitope influenza polypeptide in a solution comprising 6 M urea, 2 M thiourea, 1% CHAPS, 50 mM β-mercaptoethanol, 50 mM glycine having a pH of about 9.5;
ii. inducing aggregation by gradual removal of the chaotropic and reducing agents, thereby forming suspension of insoluble aggregates;
iii. subjecting the suspension to concentration and buffer-exchange steps by ultrafiltration comprising gradually addition of 0.1-1 M arginine buffer; and
iv. subjecting the suspension to buffer exchange to achieve a final suspension comprising about 1-5 mg/ml of the polypeptide, about 0.1-0.5 M arginine and about 10-50 mM citrate buffer, and having a pH in the range of 4 to 7.

According to other embodiments, a final suspension comprising about 2.5 mg/ml of the polypeptide, about 0.2 M arginine and about 20 mM citrate buffer, and having a pH of about 6 is achieved in step (iv).

According to other embodiments, the process comprises the steps of:
i. solubilizing inclusion bodies comprising recombinantly-produced multimeric-multiepitope influenza polypeptide in a solution comprising 6 M urea, 2 M thiourea, 1% CHAPS, 50 mM β-mercaptoethanol, 50 mM HEPES, 5 mM glycine, and having a pH of about 8.0;
ii. inducing aggregation by gradual removal of the chaotropic and reducing agents, thereby forming suspension of insoluble aggregates;
iii. subjecting the suspension to concentration and buffer-exchange steps by ultrafiltration comprising gradually addition of 20-80 mM citrate buffer; and
iv. subjecting the suspension to dilution with arginine containing solution to achieve a final uniform microparticulate suspension comprising about 1-5 mg/ml of the polypeptide, about 0.1-0.5 M arginine and about 10-50 mM citrate buffer, and having a pH in the range of 4 to 7.

According to other embodiments, a final stable and uniform microparticulate suspension comprising about 2.5 mg/ml of the polypeptide, about 0.2 M arginine and about 20 mM citrate buffer, and having a pH about 6 is achieved in step (iv).

According to some embodiments the final microparticulate suspension comprises aggregate size distribution within the range of 0.5-50 μm wherein 95% of the aggregate sizes falling into a range of one order of magnitude.

According to some embodiments 95% of the aggregates in the final microparticulate suspension have a size distribution selected from the group consisting of 0.5-5 μm, 1-10 μm, 3-30 μm and 5-50 μm. each possibility represents a separate embodiment of the present invention.

The present invention further provides a pharmaceutical composition produced according to any of the processes herein detailed.

A multimeric polypeptide according to the present invention comprises a plurality of influenza virus peptide epitopes each epitope being present at least twice in a single polypeptide. Within the context of this invention, a “multimeric” polypeptide is a polypeptide that contains a plurality of repeats (at least two, typically at least three or more), not necessarily adjacent, of an amino acid stretch of the polypeptide. The term “multimeric multiepitope” therefore relates to a polypeptide containing a plurality of repeats of a plurality of epitopes. The multimeric multiepitope polypeptide can be produced recombinantly, as an isolated polypeptide or as a fusion protein. The polypeptide aggregates can be used on their own or mixed or formulated with an additional adjuvant.

Multimeric multiepitope polypeptides contained in the pharmaceutical and vaccine compositions of the invention contain a combination of influenza virus B-cell epitopes, T-helper epitopes, and cytotoxic lymphocyte (CTL) epitopes. The epitopes are preferably selected from conserved (non-hypervariable) regions of hemagglutinin protein (HA) peptides, matrix protein (M1 and/or M2) peptides, and nucleoprotein (NP) peptides. The epitopes preferably have a demonstrable cross-reaction activity against several human influenza subtypes and are chosen for their improved ability to induce cellular and humoral immune responses.

According to other embodiments the influenza peptide epitopes within the multimeric polypeptide contained in a composition according to the present invention are selected from the group consisting of SEQ ID NO:1 to SEQ ID NO:82. Each possibility represents a separate embodiment of the present invention.

According to some specific embodiments the influenza peptide epitopes are selected from epitopes E1-E9 (SEQ ID NOs. 82, 48, 25, 52, 51, 59, 89, 69 and 70. Each possibility represents a separate embodiment of the present invention.

According to some specific embodiments a multimeric multiepitope polypeptide comprises 3-5 repeats of 4-9 epitopes arranged in a block copolymer structure or in alternating sequential polymeric structure. Each possibility represents a separate embodiment of the present invention.

According to yet other embodiments the multimeric polypeptide sequence contained in a pharmaceutical composition according to the present invention is selected from the group consisting of: SEQ ID NO:84, SEQ ID NO:86, and SEQ ID NO:88. Each possibility represents a separate embodiment of the present invention.

The pharmaceutical composition according to the present invention can be administrated via a route selected from the group consisting of: intramuscular, intranasal, oral, intraperitoneal, subcutaneous, topical, intradermal, and transdermal delivery. According to some embodiments, the pharmaceutical composition is in a form suitable for a delivery method selected from the group consisting of: intramuscular, intranasal, oral, intraperitoneal, subcutaneous, topical, intradermal, and transdermal delivery.

The pharmaceutical composition according to the present invention can be administrated via a route selected from the group consisting of: injectable, oral, intranasal, sublingual and rectal. According to some embodiments, the pharmaceutical composition is in in a form selected from injectable, oral, intranasal, sublingual and rectal. Each possibility represents a separate embodiment.

According to yet other embodiments, the pharmaceutical composition is administered via a non-injectable route. According to some embodiments, the non-injectable route of administration is selected from the group consisting of: oral, intranasal, sublingual, topical and rectal.

The pharmaceutical composition according to the present invention is administered via an intranasal route. According to some embodiments, the pharmaceutical composition is in a form suitable for an intranasal administration.

According to particular embodiments the pharmaceutical composition is administered intranasally, intramuscularly or intradermally. According to some embodiments the multimeric polypeptide contained in a pharmaceutical composition is not conjugated to and are devoid of a carrier or a fusion protein. In other embodiments a polypeptide contained in pharmaceutical compositions of the present invention may further comprise a carrier sequence, namely the peptide epitopes are inserted within a sequence of a carrier polypeptide or are coupled to a carrier sequence.

According to some embodiments the pharmaceutical compositions according to the present invention do not contain an adjuvant. According to other embodiments the vaccine further comprises a pharmaceutically acceptable adjuvant.

Pharmaceutically acceptable adjuvants include, but are not limited to water in oil emulsion, lipid emulsion, or submicron oil in water emulsion and liposomes. According to specific embodiments the adjuvant is selected from the group consisting of: Montanide™, alum, muramyl dipeptide, Gelvac®, chitin microparticles, chitosan, cholera toxin subunit B, Intralipid®, Lipofundin® or bacterial lipids, lipoproteins, and/or membrane proteins.

In some embodiments the vaccine is formulated for intramuscular, intranasal, oral, intraperitoneal, subcutaneous, topical, intradermal and transdermal delivery. In some embodiments the vaccine is administered intranasally. In other embodiments the vaccine is administered intramuscularly. In yet other embodiments the vaccine is administered intradermally.

The invention provides, according to a further aspect a method of inducing an immune response and conferring protection against influenza in a subject, comprising administering to the subject a pharmaceutical composition described above in a form of a vaccine.

Use of a vaccine composition according to the present invention, for immunization against influenza is also within the scope of the present invention. The vaccine composition according to the present invention may be administered as a stand-alone vaccine, or as part of a vaccination regiment comprising co-administration with a seasonal or pandemic influenza vaccine. Co-administration according to the present invention encompass either that both the multimeric polypeptide and the seasonal or pandemic vaccine are included in one combined composition, or that they are administered to the patient at least 24 hours apart, in two separate vaccinations.

According to some embodiments a vaccine composition comprising at least one multimeric-multiepitope influenza polypeptide is co-administered with a seasonal or pandemic influenza vaccine.

The multimeric-multiepitope polypeptides comprising a plurality of influenza virus peptide epitopes, contained in the compositions of the present invention can be produced as a recombinant protein, a fusion protein, and by chemical synthesis.

The present invention further provides pharmaceutical compositions comprising a combination of at least one multimeric influenza polypeptide and at least one conventional seasonal or pandemic influenza composition. A conventional seasonal vaccine (TIV) typically contains three inactivated or live attenuated influenza virus strains selected each year by the WHO to provide protection against the strains that are expected to infect in the coming season. A pandemic vaccine typically includes one influenza virus strain specific to the relevant strain causing the pandemic.

Further embodiments and the full scope of applicability of the present invention will become apparent from the detailed description given hereinafter. However, it should be understood that the detailed description and specific examples, while indicating particular embodiments of the invention, are given by way of illustration only, since various changes and modifications within the spirit and scope of the invention will become apparent to those skilled in the art from this detailed description.

### DETAILED DESCRIPTION OF THE INVENTION

Improved pharmaceutical compositions of multimeric multiepitope polypeptide influenza vaccines, and methods for their production are herein provided. These pharmaceutical compositions have improved stability properties. While previous compositions comprising multimeric multiepitope influenza polypeptides were based on phosphate-buffer-saline, were difficult to produce and had a tendency to re-aggregate, the present invention provides stable suspension compositions comprising the amino acid Arginine or a derivative thereof, and citrate buffer and improved processes for their production. Also provided are solid compositions comprising for example dry powder prepared from the suspensions of the invention.

In previously known preparation methods, a polypeptide aggregation was induced by a pH shift from basic to neutral pH, resulting in creation of large amorphous aggregates which required high pressure homogenization in order to convert them into injectable form. The present invention overcomes these limitations by controlled generation of protein aggregates during removal of chaotropic and reducing agents from the polypeptide solution, together with addition of the amino acid arginine, resulting in stable and uniform microparticulate suspension of the multimeric-multiepitope influenza polypeptide.

### Multimeric Multiepitope Polypeptides

A multimeric polypeptide used in vaccines and methods according to the present invention comprises at least two repeats of each epitope. A multimeric polypeptide according to the present invention comprises according to some particular embodiments, at least three repeats of each epitope. Vaccine compositions comprising a seasonal or pandemic vaccine and at least one multimeric multiepitope polypeptide, comprising a plurality of influenza virus peptide epitopes, are also provided.

Various exemplary embodiments are provided, for multimeric vaccines comprising influenza epitopes selected from Table 1, wherein the number of repeats for each epitope is the same or different and wherein the polypeptide can be arranged in an alternating sequential polymeric structure or a block copolymer structure. The term “alternating sequential polymeric” structure means that a single copy of all the epitopes contained in the polypeptide are arranged sequentially and this arrangement is repeated sequentially a number of times equal to the number of repeats. For example, if the multimeric multiepitope polypeptide comprises four repeats of three epitopes X₁, X₂ and X₃ in an alternating sequential structure, the polypeptide has the following polymeric structure: X₁X₂X₃-X₁X₂X₃-X₁X₂X₃-X₁X₂X₃, also written [X₁X₂X₃]₄. The term “block copolymer” structure means that all the copies of a single epitope contained in the polypeptide are arranged adjacently. For example, a similar multimeric multiepitope polypeptide comprising four repeats of three epitopes X₁, X₂ and X₃ in a block copolymer structure has the following polymeric structure: X₁X₁X₁X₁-X₂X₂X₂X₂-X₃X₃X₃X₃, also written [A]₄-[B]₄-[C]₄.

The synthetic or recombinant influenza multi-epitope polypeptide used in pharmaceutical compositions according to the present invention is selected from the group consisting of:

i. B(X₁ZX₂Z . . . Xₘ)ₙB; and

ii. B(X₁)ₙZ(X₂)ₙZ . . . (Xₘ)ₙB;

wherein B denotes a sequence of 0-4 amino acid residues; n is at each occurrence independently an integer of 2-50; m is an integer of 3-50; each of X₁, X₂ . . . Xₘ is an influenza peptide epitope consisting of 4-24 amino acid residues; Z at each occurrence is a bond or a spacer of 1-4 amino acid residues; and wherein the maximal number of amino acid residues in the polypeptide is about 1000. Each possibility represents a separate embodiment of the present invention.

According to some embodiments n is at each occurrence independently an integer of 2-50; m is an integer of 3-15; each of X₁-Xₘ is an influenza peptide epitope selected from the group consisting of a B-cell type epitope, a T-helper (Th) type epitope, and a cytotoxic lymphocyte (CTL) type epitope, consisting of 4-24 amino acid residues. Each possibility represents a separate embodiment of the present invention.

According to yet other embodiments, n is at each occurrence independently an integer of 2-15 and m is an integer of 3-12. Each possibility represents a separate embodiment of the present invention.

According to some specific embodiments m is an integer of 4-9 and n is an integer of 3-5. Each possibility represents a separate embodiment of the present invention.

According to some specifics embodiment m is 9 and n is 3.

According to other embodiments the influenza peptide epitopes of the multimeric polypeptide contained in a pharmaceutical composition of the invention, are selected from the group consisting of a Hemagglutinin (HA) peptide, an M1 peptide, an M2 peptide, and a nucleoprotein (NP) peptide. Each possibility represents a separate embodiment of the present invention.

According to other embodiments the influenza peptide epitopes within the multimeric polypeptide contained in a composition according to the present invention are selected from the group consisting of SEQ ID NO:1 to SEQ ID NO:82 (depicted in Table 1). Each possibility represents a separate embodiment of the present invention.

TABLE 1

| SEQ ID | |
|---|---|
| NO: | Amino Acid Sequence |
| 1 | EVET |
| 2 | MSLLTEVETHTRNGW |
| 3 | PIRNEWGCR |
| 4 | ETPIRNEWGC |
| 5 | PIRNEWGCRCN |
| 6 | LLTEVETPI |
| 7 | SLLTEVETP |
| 8 | SLLTEVETPI |
| 9 | LTEVETPLT |
| 10 | MSLLTEVETPIRNEW |
| 11 | MSLLTEVETPIRNEWGCR |
| 12 | MSLLTEVETLTKNGW |
| 13 | MSLLTEVETLTRNGW |
| 14 | LTEVETPIR |
| 15 | LTEVETPIRN |
| 16 | EVETPIRNE |
| 17 | EVETPIRNEW |
| 18 | LTEVETPIRNE |
| 19 | LTEVETPIRNEWGCR |
| 20 | EVETPIRN |
| 21 | MSLLTEVETPTRNEWECR |
| 22 | SLLTEVETPTRNEWECRCSDSSD |
| 23 | SLLTEVETPIRNEWGCRCNDSSD |
| 24 | VETPIRNEW |
| 25 | SLLTEVETYVL |
| 26 | SLLTEVETYVP |
| 27 | LLTEVETYV |
| 28 | SIVPSGPL |
| 29 | SGPLKAEIAQRLEDV |
| 30 | GPLKAEIAQRLE |
| 31 | RLEDVFAGK |
| 32 | ALMEWLKTRPI |
| 33 | PILSPLTKGI |
| 34 | ILSPLTKGI |
| 35 | LTKGILGFVFTLTVPSERG |
| 36 | TKGILGFVFTLTV |
| 37 | KGILGFVFTLTV |
| 38 | GILGFVFTL |
| 39 | LGFVFTLTV |
| 40 | ILGFVFTLT |
| 41 | ASCMGLIY |
| 42 | RMGAVTTEV |
| 43 | GLVCATCEQIA |
| 44 | QMVATTNPL |
| 45 | QMVATTNPLI |
| 46 | RMVLASTTAK |
| 47 | DLLENLQTY |
| 48 | SKAYSNCYPYDVPDYASL |
| 49 | SKAFSNCYPYDVPDYASL |
| 50 | STAYSNCYPYDVPDYASL |
| 51 | WTGVTQN |
| 52 | WLTGKNGLYP |
| 53 | WLTEKEGSYP |
| 54 | PKYVKQNTLKLATGMRNVP |
| 55 | GVKLESMGIYQ |
| 56 | EISGVKLESMG |
| 57 | NVKNLYEKVK |
| 58 | KVKILPKDRWTQHTTTGG |
| 59 | PKYVKQNTLKLAT |
| 60 | KTGGPIYRR |
| 61 | CTELKLSDY |
| 62 | HPSAGKDPKKTGGP |
| 63 | HPSAGKDPKKTGG |
| 64 | FWRGENGRKTRSAYERMCNILKGK |
| 65 | ILRGSVAHK |
| 66 | KLLQNSQVY |
| 67 | SAAFEDLRVLSFIRG |
| 68 | SAAFEDLRVSSFIRGT |
| 69 | SAAFEDLRVLSFIRGY |
| 70 | ELRSRYWAIRTRSG |
| 71 | ELRSRYWAI |
| 72 | SRYWAIRTR |
| 73 | YWAIRTRSGG |
| 74 | SRYWAIRTR |
| 75 | LPFDKPTIM |
| 76 | VSDGGPNLY |
| 77 | RRSFELKKL |
| 78 | RRATAILRK |
| 79 | RPIIRPATL |
| 80 | ADRGLLRDI |
| 81 | PYYTGEHAKAIGN |
| 82 | PAKLLKERGFFGAIAGFLE |

According to some specific embodiments the influenza peptide epitopes are selected from epitopes E1-E9 (SEQ ID NOs. 82, 48, 25, 52, 51, 59, 89, 69 and 70, detailed in Table 2.

TABLE 2
influenza peptide epitopes E1 to E9

| | Epitope | Protein | Amino Acid | SEQ ID |
|---|---|---|---|---|
| Epitope | Type | residues | Sequence | NO: |
| E1 | B cell | HA 354-372 | PAKLLKERGFFGAIAGFLE | 82 |
| E2 | B cell | HA 91-108 | SKAYSNCYPYDVPDYASL | 48 |
| E3 | B cell & CTL | M1 2-12 | SLLTEVETYVL | 25 |
| E4 | B cell | HA 150-159 | WLTGKNGLYP | 52 |
| E5 | B cell | HA 143-149 | WTGVTQN | 51 |
| E6 | T helper | NP 206-229 | FWRGENGRKTRSAYERMCNILKGK | 64 |
| E7 | T helper | HA 307-319 | PK/RYVKQNTLKLAT | 59, 89 |
| E8 | CTL | NP 335-350 | SAAFEDLRVLSFIRGY | 69 |
| E9 | CTL | NP 380-393 | ELRSRYWAIRTRSG | 70 |

It is to be noted that peptide epitopes listed herein are provided as for exemplary purposes only. The influenza virus proteins vary between isolates, thereby providing multiple variant sequences for each influenza protein. Accordingly, the present invention encompasses peptide epitopes having one or more amino acid substitutions, additions or deletions.

According to more specific embodiments the influenza peptide epitopes included in a multimeric polypeptide contained in a vaccine pharmaceutical composition according to the present invention consist of: HA 354-372 (E1, SEQ ID NO: 82), HA 91-108 (E2, SEQ ID NO: 48), M1 2-12 (E3, SEQ ID NO: 25), HA 150-159 (E4, SEQ ID NO: 52), HA 143-149 (E5, SEQ ID NO: 51), NP 206-229 (E6, SEQ ID NO: 64), HA 307-319 (E7, SEQ ID NO: 59 or 89), NP 335-350 (E8, SEQ ID NO: 69), and NP 380-393 (E9, SEQ ID NO: 70).

According to yet other embodiments the multimeric polypeptide sequence contained in a pharmaceutical composition according to the present invention is selected from the group consisting of: SEQ ID NO:84, SEQ ID NO:86, and SEQ ID NO:88. Each possibility represents a separate embodiment of the present invention.

According to yet other embodiments the multimeric polypeptide sequence contained in a pharmaceutical composition according to the present invention is encoded by a polynucleotide sequence selected from the group consisting of: SEQ ID NO:83, SEQ ID NO:85, and SEQ ID NO:87. Each possibility represents a separate embodiment of the present invention.

In some embodiments the pharmaceutical composition comprises three to five repeats of the five to nine epitopes arranged in a block copolymer structure or in alternating sequential polymeric structure. Each possibility represents a separate embodiment of the present invention.

According to s a pharmaceutical composition of the present invention comprises three repeats of nine different influenza virus peptide epitopes arranged in the following block copolymer structure [E1E1E1-E2E2E2-E3E3E3-E4E4E4-E5E5E5-E6E6E6-E7E7E7-E8E8E8-E9E9E9], wherein E1 is HA 354-372 (SEQ ID NO:82), E2 is HA 91-108 (SEQ ID NO:48), E3 is M1 2-12 (SEQ ID NO:25), E4 is HA 150-159 (SEQ ID NO:52), E5 is HA 143-149 (SEQ ID NO:51), E6 is NP 206-229 (SEQ ID NO:64), E7 is HA 307-319 (SEQ ID NO:59 or 89), E8 is NP 335-350 (SEQ ID NO:69), and E9 is NP 380-393 (SEQ ID NO:70).

According to other embodiments the multimeric polypeptide comprises nine different influenza virus peptide epitopes arranged in the following alternating sequential polymeric structure [E1E2E3E4E5E6E7E8E9]ₙ, wherein n is 3 to 5; E1 is HA 354-372 (SEQ ID NO:82), E2 is HA 91-108 (SEQ ID NO:48), E3 is M1 2-12 (SEQ ID NO:25), E4 is HA 150-159 (SEQ ID NO:52), E5 is HA 143-149 (SEQ ID NO:51), E6 is NP 206-229 (SEQ ID NO:64), E7 is HA 307-319 (SEQ ID NO:59 or 89), E8 is NP 335-350 (SEQ ID NO:69), and E9 is NP 380-393 (SEQ ID NO:70).

According to yet other embodiments the multimeric polypeptide comprises six repeats of five different B-cell type influenza virus peptide epitopes arranged in the following alternating sequential polymeric structure [E1E2E3E4E5]₆, wherein E1 is HA 354-372 (SEQ ID NO:82), E2 is HA 91-108 (SEQ ID NO:48), E3 is M1 2-12 (SEQ ID NO:25), E4 is HA 150-159 (SEQ ID NO:52), E5 is HA 143-149 (SEQ ID NO:51).

According to other embodiments the multimeric polypeptide comprises six repeats of four different T-cell type influenza virus peptide epitopes arranged in the following alternating sequential polymeric structure [E7E8E9E6]₆, wherein E6 is NP 206-229 (SEQ ID NO:64), E7 is HA 307-319 (SEQ ID NO:59 or 89), E8 is NP 335-350 (SEQ ID NO:69), and E9 is NP 380-393 (SEQ ID NO:70).

According to additional embodiments the multimeric polypeptide comprises six repeats of nine different influenza virus peptide epitopes arranged in the following block copolymer structure [E2E2E2E2E2E2-E1E1E1E1E1E1-E3E3E3E3E3E3-E4E4E4E4E4E4-E5E5E5E5E5E5-E6E6EE6E6E6-E7E7E7E7E7E7-E8E8E8E8E8E8-E9E9E9E9E9E9], wherein E1 is HA 354-372 (SEQ ID NO:82), E2 is HA 91-108 (SEQ ID NO:48), E3 is M1 2-12 (SEQ ID NO:25), E4 is HA 150-159 (SEQ ID NO:52), E5 is HA 143-149 (SEQ ID NO:51), E6 is NP 206-229 (SEQ ID NO:64), E7 is HA 307-319 (SEQ ID NO:59 or 89), E8 is NP 335-350 (SEQ ID NO:69), and E9 is NP 380-393 (SEQ ID NO:70).

In various embodiments the multimeric polypeptide comprises at least two repeats of each epitope, typically at least three repeats of each epitope, alternatively at least four repeats, alternatively at least five repeats, alternatively at least six repeats of each epitope, maximum at least 50 repeats of each epitope. To improve the exposure of the epitopes to the immune system, the epitopes may be separated by a spacer, which according to certain embodiments consists of a single amino acid and according to other embodiments comprises 2-6 amino acids. According to some specific embodiments, the spacer consists of 1-4 neutral amino acid residues. Each possibility represents a separate embodiment of the present invention.

According to some embodiments peptide epitopes within a multimeric polypeptide contained in a pharmaceutical composition are linked by a spacer selected from the group consisting of: a bond, an amino acid, and a peptide comprising 2-6 amino acids.

According to some embodiments at least one amino acid of the spacer induces a specific conformation on a segment of the polypeptide (e.g. a proline residue).

According to yet other embodiments the spacer comprises a cleavable sequence. According to one embodiment the cleavable spacer is cleaved by intracellular enzymes. According to a more specific embodiment the cleavable spacer comprises a protease specific cleavable sequence.

### Processes for Producing Suspensions

### Solubilization of ‘Insoluble’ Inclusion Bodies

Inclusion bodies of recombinant multimeric-multiepitope polypeptides formed in two different strains of E. coli exhibited limited solubility, probably due to hydrophobic interactions. Standard procedure of 8 M urea buffer supplemented with 50 mM βME did not solubilize the inclusion bodies at neutral pH. Addition of non-ionic or zwitterionic detergents did not improve the buffer performance. Anionic detergent addition (sodium dodecyl sulfate, SDS) enabled complete inclusion bodies solubilization, but this method is unsuitable due to SDS interference with following purification steps. Initially, basic (12) pH was used in order to solubilize the inclusion bodies in 8 M urea buffer. Later on, in was surprisingly found that a buffer regularly used for membrane protein solubilization can be modified and used for inclusion bodies solubilization at a lower pH (8-9.5). A solution comprising 6 M urea, 2 M thiourea, 1% CHAPS (3[(3-cholamidopropyl)dimethylammonio]-1-propanesulfonate), 50 mM βME, 50 mM glycine and a pH of about 9.5 was used to achieve full solubilization of the inclusion bodies.

### Ultrafiltration and Aggregate Formation

Ultrafiltration is used, according to the present invention, in the production process of the multimeric-multiepitope polypeptides, for gradual removal of chaotropic (urea and thiourea for example) and reducing (βME for example) agents, and gradual addition of arginine, for controlled creation of protein aggregates at a protein concentration of 1-50 mg/ml, suitable for use, after buffer exchange, in the final composition.

### Arginine Stabilizing Effect

Arginine is not used, according to the present invention, to prevent aggregate formation as described in the art, but for size reduction and increased uniformity of protein microparticles. High concentration (about 0.2-1 M) of arginine buffer is added gradually to solubilized inclusion bodies, via an ultrafiltration buffer exchange process. The final arginine concentration of 0.1-0.5 M is achieved by further buffer exchange.

### Suspension Compositions

As disclosed by Akers et al., (J. Parenteral Sci. Technol., 41, 88-96, 1987), the basic reasons for preparing pharmaceutical suspensions include the following:

1. The solubility of the peptide or protein prohibits solution formulation.

2. The stability of the peptide or protein is improved in a suspension formulation.

3. There is a desire to control or retard the release profile of the peptide or protein.

There are at least four approaches for producing particles that can be considered for development of peptide or protein suspension formulations:

1. The suspension is composed exclusively of crystalline material in vehicle.

2. The suspension is composed exclusively of amorphous material in vehicle.

3. The suspension contains a mixture of crystalline and amorphous material in vehicle.

4. The suspension contains active ingredient in both the suspension and solution phases.

### Solid Compositions

There are at least four methods of producing the sterile powder: crystallization, lyophilization, spray-drying, and supercritical fluid particle formation. Given the propensity of peptides and proteins to denature under high-stress conditions, crystallization and lyophilization are more generally applicable to this class of compounds. These two methods also have proven ability to maintain the sterility of the dried material. The spray-drying process might result in denaturation either at the liquid-air interface or from the high temperature required to evaporate solvent; however, the technique may be appropriate for small peptides that lack higher-order structure.

### Lyophilized Compositions

Lyophilization, or freeze drying is a drying process, which entails freezing a liquid dispersion, such as an aqueous solution or suspension and removing the liquid material (e.g. the solvent) using reduced pressure. Specifically, lyophilized composition may be formed from aqueous suspensions through freezing the aqueous suspensions to form a heterogeneous solid mixture of the frozen ice and the aggregates, and then lowering the pressure, thereby removing the ice by sublimation and maintaining the aggregate material. Lyophilization results in a high-quality product because of the low temperature used in processing. Thus, the original shape of the product is maintained and quality of the rehydrated product may be improved compared to mare conventional evaporation or distillation at liquid water temperature.

In the context of vaccinations, freeze dried solid pharmaceutical composition, such as those in the form of a lyophilized powder are especially beneficial for vaccine administration in intranasal routes and for improved stability and longer shelf life.

According to some embodiments, the present invention provides a solid pharmaceutical composition prepared by lyophilizing the pharmaceutical composition in the form of an aqueous suspension of microparticles disclosed herein.

According to some embodiments, the solid pharmaceutical composition is in the form of a powder. According to some embodiments, the solid pharmaceutical composition is in the form of a dry powder.

A “powder” as used herein, refers to a substantially dry, bulk solid composed of a large number of particles. Powders are a sub-class of granular materials.

The term “dry powder” is intended to denote a solid whose water content is generally less than or equal to 10% by weight. This content is preferably less than or equal to 5% by weight, and most particularly less than 2% by weight.

According to some embodiments, the present invention further provides a pharmaceutical composition in the form of an aqueous suspension of microparticles, said composition is prepared by suspending the solid composition disclosed herein in a diluent. According to some embodiments, the diluent is water.

Aqueous or non-aqueous vehicle containing any necessary excipients is prepared separately—from particle formation. Examples of non-aqueous vehicles include any highly purified natural or synthetic oil such as sesame, peanut, or other vegetable oils. Depending on the solubility of the constituents and overall viscosity of the vehicle, sterilization can be accomplished by either filtration or autoclaving. The sterile combination approach offers more flexibility in the choice of vehicle (aqueous or non-aqueous), since particle growth is accomplished independently. Once processing of each section is completed, the dry particles and vehicle are aseptically combined. Some form of agitation is required to achieve a homogeneous dispersion of particles. In the case of peptides or proteins, appropriate controls should be in place to ensure that the dispersion process does not result in denaturation or other physical changes.

The present invention provides a process of producing a purified recombinant multimeric-multiepitope polypeptide suspension, comprising the steps:
i. solubilizing inclusion bodies containing at least one multimeric-multiepitope polypeptide in a solution comprising a chaotropic agent, a buffering agent, and a reducing agent, having a pH in a range of 7-11; and
ii. inducing controlled aggregation by gradual removal of the chaotropic and reducing agents consequently forming insoluble aggregate suspension;
iii. addition of arginine to achieve a stable and uniform suspension having a uniform aggregate size distribution, with 95% of the aggregate sizes falling into a size range of one order of magnitude.

According to some embodiments the pH in step (i) is in a range of 7-10.

According to some embodiments the chaotropic agent is urea, thiourea, or a combination thereof.

According to specific embodiments the chaotropic agent comprises 5-8 M urea and 1-4 M thiourea.

According to particular embodiments, gradual removal of the chaotropic and reducing agents in step (ii) is performed by ultrafiltration.

According to some embodiments, the CHAPS in step (i) is present at a concentration of 0.5-2%.

According to some embodiments, the process further comprises the steps of
iv. freezing the stable microparticulate suspension of step (iii) at a temperature in the range of −10° C. to −50° C.; and
v. reducing the pressure of the frozen composition of step (iv) for a period of time, so as to sublime at least 95% of the water contained therein, thereby providing a solid composition of the multimeric multiepitope influenza polypeptide.

According to some embodiments, step (v) comprises reducing the pressure of the frozen composition of step (iv) for a period of 24 hours to 72 hours, so as to sublime at least 95% of the water contained therein, thereby providing the solid composition of the multimeric multiepitope influenza polypeptide. According to some embodiments, step (v) comprises reducing the pressure of the frozen composition of step (iv) for a period of 24 hours to 72 hours, so as to sublime at least 98% of the water contained therein, thereby providing the solid composition of the multimeric multiepitope influenza polypeptide. According to some embodiments, step (v) comprises reducing the pressure of the frozen composition of step (iv) for a period of 24 hours to 72 hours, so as to sublime at least 99% of the water contained therein, thereby providing the solid composition of the multimeric multiepitope influenza polypeptide. According to some embodiments, step (v) comprises reducing the pressure of the frozen composition of step (iv) for a period of 24 hours to 72 hours, so as to sublime at least 99.5% of the water contained therein, thereby providing the solid composition of the multimeric multiepitope influenza polypeptide.

According to some embodiments, step (i) comprises providing at least one multimeric multiepitope influenza polypeptide at a concentration of 1.5-5 mg/ml in an aqueous solution comprising a chaotropic agent, a detergent, a reducing agent, and a buffering agent providing a pH in the range of 7-10.

According to some embodiments, the chaotropic agent is 5-8 M urea and 1-4 M thiourea, the detergent is 0.5-4% CHAPS (3-[(3-cholamidopropyl) dimethylammonio]-1-propanesulfonate) and the buffering agent is 5-100 mM glycine, and wherein the guanidinium-containing amino acid is an arginine (Arg).

According to some embodiments, the present invention provides a solid pharmaceutical composition of multimeric multiepitope influenza polypeptide, comprising a solid composition of the multimeric multiepitope influenza polypeptide prepared according to the process disclosed herein, and a pharmaceutically-acceptable excipient or carrier.

According to some embodiments, the solid pharmaceutical composition is in the form of a powder. According to some embodiments, the solid pharmaceutical composition is in the form of a dry powder.

According to some embodiments, the process further comprises re-suspending the solid composition of step (v) in water, thereby providing a pharmaceutical composition in the form of an aqueous suspension of microparticles.

According to some embodiments, there is provided a packaged pharmaceutical composition comprised of a packaging material; and the solid pharmaceutical composition of the present invention. According to some embodiments, the packaged pharmaceutical composition further comprises a diluent to be used for solubilizing the solid composition.

According to some embodiments, the process comprises the steps:
i. providing E. coli cells expressing at least one multimeric-multiepitope influenza polypeptide;
ii. performing lysis and bacterial cell disruption to provide inclusion bodies comprising multimeric-multiepitope influenza polypeptide;
iii. solubilizing the inclusion bodies in a solution comprising 5-8M urea, 1-4 M thiourea, 0.5-4% CHAPS (3-[(3-cholamidopropyl)dimethylammonio]-1-propanesulfonate), a buffering agent, and a reducing agent, at a pH in the range of 7-11;
iv. inducing controlled aggregation by gradual removal of the chaotropic and reducing agents consequently forming insoluble aggregate suspension;
v. addition of arginine to achieve a stable and uniform suspension having a uniform aggregate size distribution, with 95% of the aggregates falling into a size range of one order of magnitude.

According to some embodiments, the process comprises the steps:
i. providing E. coli cells expressing at least one multimeric-multiepitope influenza polypeptide;
ii. performing lysis and bacterial cell disruption to provide inclusion bodies comprising multimeric-multiepitope influenza polypeptide;
iii. solubilizing the inclusion bodies in a solution comprising 5-8M urea, 1-4 M thiourea, 0.5-4% CHAPS (3-[(3-cholamidopropyl)dimethylammonio]-1-propanesulfonate), a buffering agent, and a reducing agent, at a pH in the range of 7-11,
iv. inducing controlled aggregation by gradual removal of the chaotropic and reducing agents consequently forming insoluble aggregate suspension;
v. addition of arginine to achieve a stable and uniform suspension having a uniform aggregate size distribution, with 95% of the aggregates falling into a size range of one order of magnitude,
vi. freezing the uniform suspension of step (v) at a temperature in the range of −10° C. to −50° C.; and
vii. reducing the pressure of the frozen composition of step (vi) for a period of time, so as to sublime at least 95% of the water contained therein, thereby providing a solid composition of the multimeric multiepitope influenza polypeptide.

According to some embodiments, gradual removal of chaotropic and reducing agents is performed by ultrafiltration using a procedure known in the art.

According to some embodiments, at least one chromatography separation step is performed between steps (iii) and (iv). According to some embodiments, the at least one chromatography separation step is selected from the group consisting of ion exchange chromatography and hydrophobic interaction chromatography.

According to some embodiments, step (v) is performed prior to aggregate formation of step (iv).

According to other embodiments, step (v) is performed during aggregate formation of step (iv).

According to yet other embodiments, step (v) is performed following aggregate formation of step (iv).

According to some embodiments, the process comprises:
i. providing E. coli cells expressing at least one multimeric-multiepitope influenza polypeptide;
ii. performing lysis, bacterial cell disruption and centrifugation of the E. coli cells to provide inclusion bodies comprising multimeric-multiepitope influenza polypeptide;
iii. performing at least one wash of the inclusion bodies;
iv. solubilizing the inclusion bodies in a solution comprising 5-8M urea, 1-4 M thiourea, 0.5-4% CHAPS (3-[(3-cholamidopropyl)dimethylammonio]-1-propanesulfonate), a reducing agent and a buffering agent providing a pH in the range of 7-10;
v. subjecting the solution to at least one chromatography step;
vi. inducing of controlled aggregation by gradual removal of the chaotropic and reducing agents, thereby forming insoluble aggregates;
vii. addition of guanidinium-containing compound, and
viii. formulating the composition of (vii) to achieve a final suspension of about 0.1-0.4 M guanidinium-containing amino acid and 10-40 mM citrate buffer, at a pH in the range of 4-8.

According to some embodiments, the process comprises:
i. providing E. coli cells expressing at least one multimeric-multiepitope influenza polypeptide;
ii. performing lysis, bacterial cell disruption and centrifugation of the E. coli cells to provide inclusion bodies comprising multimeric-multiepitope influenza polypeptide;
iii. performing at least one wash of the inclusion bodies;
iv. solubilizing the inclusion bodies in a solution comprising 5-8M urea, 1-4 M thiourea, 0.5-4% CHAPS (3-[(3-cholamidopropyl)dimethylammonio]-1-propanesulfonate), a reducing agent and a buffering agent providing a pH in the range of 7-10;
v. subjecting the solution to at least one chromatography step;
vi. inducing of controlled aggregation by gradual removal of the chaotropic and reducing agents, thereby forming insoluble aggregates;
vii. addition of guanidinium-containing compound,
viii. formulating the composition of (vii) to achieve a final suspension of about 0.1-0.4 M guanidinium-containing amino acid and 10-40 mM citrate buffer, at a pH in the range of 4-8;
ix. freezing the final of step (viii) at a temperature in the range of −10° C. to −50° C.; and
x. reducing the pressure of the frozen composition of step (vi) for a period of time, so as to sublime at least 95% of the water contained therein, thereby providing a solid composition of the multimeric multiepitope influenza polypeptide.

According to other embodiments the addition of guanidinium-containing compound of step (vii) is performed gradually during induction of controlled aggregation.

According to some embodiments, the bacterial cell disruption of step (ii) is performed by high pressure homogenization.

According to other embodiments, the bacterial cell disruption is performed by sonication.

According to some embodiments, lysis is performed in a buffer comprising Tris buffer, EDTA, and 0.1-0.5 mg lysozyme per 1 g of cell paste at a pH in the range of 7-9.

According to some embodiments, the urea of step (iv) is in a concentration of 6-7 M.

According to some embodiments, the buffering agent of step (iv) is HEPES in a concentration of 20-100 mM.

According to other embodiments, the buffering agent of step (iv) is glycine in a concentration of 5-100 mM.

According to some embodiments, the reducing agent of step (iv) is β-mercaptoethanol in a concentration of 20-80 mM.

According to some embodiments, inducing of controlled aggregation of step (vi) is performed by ultrafiltration.

Any ultrafiltration method known is the art is suitable for use according to the present invention, including but not limited to hollow fiber, Tangential Flow Filtration (TFF) cassette, and stirred cell.

According to some embodiments, ultrafiltration comprises the steps of:
i. Buffer exchange I with 10-80 mM MES buffer pH 4-6.5;
ii. Buffer exchange II with 20-80 mM citrate buffer, 0.1-1 M arginine, pH 4-6;
iii. Buffer exchange III with 10-50 mM citrate buffer, 0.1-0.5 M arginine, pH 4-7.5.

According to other embodiments, ultrafiltration comprises the steps of:
i. Buffer exchange I with 10-80 mM MES buffer pH 4-6.5;
ii. Buffer exchange II with 20-80 mM citrate buffer pH 4-6;
iii. Dilution with arginine containing solution to 0.1-1 M arginine concentration.

According to some embodiments, the polypeptide concentration in the final composition is 1-10 mg/ml.

According to yet other embodiments, the polypeptide concentration in the final composition is 1-4 mg/ml.

According to some embodiments, the at least one multimeric-multiepitope influenza polypeptide sequence is set forth in SEQ ID NO. 86.

According to some embodiments, the at least one multimeric-multiepitope influenza polypeptide is encoded by a polynucleotide sequence set forth in SEQ ID NO. 85.

According to some specific embodiments the process comprises the steps:
i. solubilizing inclusion bodies comprising recombinantly-produced multimeric-multiepitope influenza polypeptide in a solution comprising 6 M urea, 2 M thiourea, 1% CHAPS, 50 mM β-mercaptoethanol, 50 mM glycine, having a pH of about 9.5;
ii. inducing aggregation by gradual removal of the chaotropic and reducing agents (urea, thiourea and β-mercaptoethanol), thereby forming suspension of insoluble aggregates;
iii. subjecting the suspension to concentration and buffer-exchange steps by ultrafiltration comprising gradually addition of 0.5 M arginine buffer; and
iv. subjecting the suspension to buffer exchange to achieve a final composition comprising about 2.5 mg/ml of the polypeptide, about 0.2 M arginine and about 20 mM citrate buffer, having a pH about 6.

According to some specific embodiments the process comprises the steps:
i. solubilizing inclusion bodies comprising recombinantly-produced multimeric-multiepitope influenza polypeptide in a solution comprising 6 M urea, 2 M thiourea, 1% CHAPS, 50 mM β-mercaptoethanol, 50 mM glycine, having a pH of about 9.5;
ii. inducing aggregation by gradual removal of the chaotropic and reducing agents (urea, thiourea and β-mercaptoethanol), thereby forming suspension of insoluble aggregates;
iii. subjecting the suspension to concentration and buffer-exchange steps by ultrafiltration comprising gradually addition of 0.5 M arginine buffer; and
iv. subjecting the suspension to buffer exchange to achieve a final composition comprising about 2.5 mg/ml of the polypeptide, about 0.2 M arginine and about 20 mM citrate buffer, having a pH about 6;
v. freezing the final composition of step (iv) at a temperature in the range of −10° C. to −50° C.; and
vi. reducing the pressure of the frozen composition of step (v) for a period of time, so as to sublime at least 95% of the water contained therein, thereby providing a solid composition of the multimeric multiepitope influenza polypeptide.

According to yet another aspect, the present invention provides a process of producing an essentially stable aqueous suspension of a recombinant multimeric-multiepitope polypeptide, comprising the steps of:
i. solubilizing inclusion bodies comprising recombinantly-produced multimeric-multiepitope influenza polypeptide in a solution comprising 6 M urea, 2 M thiourea, 1% CHAPS, 50 mM β-mercaptoethanol, 5-50 mM glycine, having a pH in the range of 7-10;
ii. inducing aggregation by gradual removal of the chaotropic and reducing agents, thereby forming suspension of insoluble aggregates;
iii. subjecting the suspension to concentration and buffer-exchange steps by ultrafiltration comprising gradually addition of an arginine buffer or a citrate buffer; and
iv. subjecting the suspension to buffer exchange to achieve a final stable and uniform suspension comprising about 1-5 mg/ml of the polypeptide, about 0.1-0.5 M arginine and about 10-50 mM citrate buffer, having a pH in the range of 4-7.

According to some embodiments, in step (i) the pH is in the range of 8-10.

According to some embodiments, the process comprises the steps:
i. solubilizing inclusion bodies comprising recombinantly-produced multimeric-multiepitope influenza polypeptide in a solution comprising 6 M urea, 2 M thiourea, 1% CHAPS, 50 mM β-mercaptoethanol, 50 mM glycine having a pH of about 9.5;
ii. inducing aggregation by gradual removal of the chaotropic and reducing agents, thereby forming suspension of insoluble aggregates;
iii. subjecting the suspension to concentration and buffer-exchange steps by ultrafiltration comprising gradually addition of 0.1-1 M arginine buffer; and
iv. subjecting the suspension to buffer exchange to achieve a final suspension comprising about 1-5 mg/ml of the polypeptide, about 0.1-0.5 M arginine and about 10-50 mM citrate buffer, and having a pH in the range of 4-7.

According to some embodiments, the process comprises the steps:
i. solubilizing inclusion bodies comprising recombinantly-produced multimeric-multiepitope influenza polypeptide in a solution comprising 6 M urea, 2 M thiourea, 1% CHAPS, 50 mM β-mercaptoethanol, 50 mM glycine having a pH of about 9.5;
ii. inducing aggregation by gradual removal of the chaotropic and reducing agents, thereby forming suspension of insoluble aggregates;
iii. subjecting the suspension to concentration and buffer-exchange steps by ultrafiltration comprising gradually addition of 0.1-1 M arginine buffer;
iv. subjecting the suspension to buffer exchange to achieve a final suspension comprising about 1-5 mg/ml of the polypeptide, about 0.1-0.5 M arginine and about 10-50 mM citrate buffer, and having a pH in the range of 4-7;
v. freezing the final suspension of step (iv) at a temperature in the range of −10° C. to −50° C.; and
vi. reducing the pressure of the frozen composition of step (v) for a period of time, so as to sublime at least 95% of the water contained therein, thereby providing a solid composition of the multimeric multiepitope influenza polypeptide.

According to some specific embodiments, in step (iii) 0.5 M arginine buffer is added;

According to other embodiments, a final suspension comprising about 2.5 mg/ml of the polypeptide, about 0.2 M arginine and about 20 mM citrate buffer, and having a pH of about 6 is achieved in step (iv).

According to other embodiments, the process comprises the steps of:
i. solubilizing inclusion bodies comprising recombinantly-produced multimeric-multiepitope influenza polypeptide in a solution comprising 6 M urea, 2 M thiourea, 1% CHAPS, 50 mM β-mercaptoethanol, 50 mM HEPES, 5 mM glycine, and having a pH of about 8.0;
ii. inducing aggregation by gradual removal of the chaotropic and reducing agents, thereby forming suspension of insoluble aggregates;
iii. subjecting the suspension to concentration and buffer-exchange steps by ultrafiltration comprising gradually addition of 20-80 mM citrate buffer; and
iv. subjecting the suspension to dilution with arginine containing solution to achieve a final uniform microparticulate suspension comprising about 1-5 mg/ml of the polypeptide, about 0.1-0.5 M arginine and about 10-50 mM citrate buffer, and having a pH in the range of 4-7.

According to other embodiments, the process comprises the steps of:
i. solubilizing inclusion bodies comprising recombinantly-produced multimeric-multiepitope influenza polypeptide in a solution comprising 6 M urea, 2 M thiourea, 1% CHAPS, 50 mM β-mercaptoethanol, 50 mM HEPES, 5 mM glycine, and having a pH of about 8.0;
ii. inducing aggregation by gradual removal of the chaotropic and reducing agents, thereby forming suspension of insoluble aggregates;
iii. subjecting the suspension to concentration and buffer-exchange steps by ultrafiltration comprising gradually addition of 20-80 mM citrate buffer;
iv. subjecting the suspension to dilution with arginine containing solution to achieve a final uniform microparticulate suspension comprising about 1-5 mg/ml of the polypeptide, about 0.1-0.5 M arginine and about 10-50 mM citrate buffer, and having a pH in the range of 4-7;
v. freezing the final uniform microparticulate suspension of step (iv) at a temperature in the range of −10° C. to −50° C.; and
vi. reducing the pressure of the frozen composition of step (v) for a period of time, so as to sublime at least 95% of the water contained therein, thereby providing a solid composition of the multimeric multiepitope influenza polypeptide.

According to some specific embodiments, in step (iii) 50 mM citrate buffer is added.

According to other embodiments, a final stable and uniform microparticulate suspension comprising about 2.5 mg/ml of the polypeptide, about 0.2 M arginine and about 20 mM citrate buffer, and having a pH about 6 is achieved in step (iv).

According to some embodiments the final microparticulate suspension comprises aggregate size distribution within the range of 0.5-50 μm wherein 95% of the aggregate sizes falling into a range of one order of magnitude.

According to some embodiments 95% of the aggregates in the final microparticulate suspension have a size distribution of 0.5-5 μm.

According to some embodiments 95% of the aggregates in the final microparticulate suspension have a size distribution of 1-10 μm.

According to some embodiments 95% of the aggregates in the final microparticulate suspension have a size distribution of 3-30 μm.

According to some embodiments 95% of the aggregates in the final microparticulate suspension have a size distribution of 5-50 μm.

### Definitions

For convenience, certain terms employed in the specification, examples and claims are described herein.

A multimeric-multiepitope polypeptide according to the present invention denotes a polypeptide comprising multiple copies of plurality of influenza virus peptide epitopes.

The term “immunogenicity” or “immunogenic” relates to the ability of a substance to stimulate or elicit an immune response. Immunogenicity is measured, for example, by determining the presence of antibodies specific for the substance. The presence of antibodies is detected by methods known in the art, for example using an ELISA or HAI assay.

Influenza epitopes can be classified as B-cell type, T-cell type or both B cell and T cell type, depending on the type of immune response they elicit. The definition of B cell or T cell peptide epitope is not unequivocal; for example, a peptide epitope can induce antibody production but at the same time that epitope can possess a sequence that enables binding to the human HLA molecule, rendering it accessible to CTLs or Th cells, hence a dual B cell and T cell classification for that particular epitope. “CTL”, “killer T cells” or “cytotoxic T cells” is a group of differentiated T cells that recognize and lyse target cells bearing a specific foreign antigen that function in defense against viral infection and cancer cells. “T helper cell” or “Th” is any of the T cells that when stimulated by specific antigen release cytokines that promote the activation and function of B cells and killer T cells.

“Amino acid sequence”, as used herein, refers to an oligopeptide, peptide, polypeptide, or protein sequence, and fragment thereof, and to naturally occurring or synthetic molecules.

In the specification and in the claims the term “spacer” denotes any chemical compound, which may be present in the polypeptide sequence, at one of the terminals or between two epitopes. According to some embodiments, the spacer consists of 1-4 amino acid residues. The spacer may comprise a sequence that can be cleaved by enzymatic means, or may decompose spontaneously. The spacer may enforce or induce beneficial conformation to the polypeptide. The spacer may optionally comprise a protease specific cleavable sequence.

The term “insoluble,” is defined herein as the characteristic of being visible to the unaided eye, when in an aqueous suspension. “Insoluble” particles can be precipitated or recovered upon centrifugation of an aqueous suspension.

The term “suspension,” is defined herein as an aqueous medium that comprises an “insoluble” particle component as defined above.

The term “microparticle” is defined herein as an “insoluble” protein aggregate of 0.1-100 μm size. According to some embodiments the microparticles of the invention are within size range of 0.5-50 μm.

The term “solution,” is defined herein as an aqueous medium that is substantially free of “insoluble” particles as that term is defined above.

“Uniformity” is defined herein by size distribution of aggregates as measured by a parameter which is tested for example by a LUMiSizer suspension analyzer (LUM GmbH). A uniform suspension is defined herein as having aggregate size distribution with 95% of the aggregate sizes falling into a range of one order of magnitude.

A uniform microparticulate suspension is an aqueous medium comprising “insoluble” protein aggregates (defined above as microparticles), having a uniform aggregate size distribution, with 95% of the aggregate sizes falling into a range of one order of magnitude (e.g. 0.1-1 μm, 1-10 μm, 3-30 μm, 9-90 μm etc.).

The size distribution of the particles contained in a uniform microparticulate suspension according to the present invention is narrow. The term “narrow particle size distribution” as used herein refers to a distribution wherein more than 90% of the particles have a particle size in the range of 0.2-2 times the mean (or average) particle size. Preferably, more than 95% of the particles have a particle size within this range. Even more preferably more than 99% of the particles have a particle size within this range. Thus, for a particle size of 5 μm, a narrow size distribution refers to a distribution wherein more than 90%, 95% or 99% of the particles have a particle size in the range of 1-10 μm.

The microparticles are essentially spherical or spheroidal in shape but non-spherical shapes of particles, including irregular shapes, are also possible. For spherical particles the size represents the diameter while for non-spherical particles the size represents the longest dimension of an average particle.

According to the principles of the present invention, the mean particle size as used herein refers to the average particle diameter which can be determined using techniques known to those of skill in the art including, but not limited to, sedimentation flow fractionation, photon correlation spectroscopy, light scattering, electron scattering, disk centrifugation, and the like. According to some embodiments, the aggregate size distribution is determined using a LUMiSizer suspension analyzer (LUM GmbH) or any corresponding equipment.

“Stability” is defined herein as the condition in which aggregate size remains uniformly distributed.

A chaotropic agent is a denaturant, namely a compound that can disrupt the hydrogen bonding network between water molecules and reduce the stability of the native state of macromolecules (e.g. proteins in the present invention) by weakening the hydrophobic effect.

A guanidinium-containing amino acid according to the present invention includes but is not limited to an arginine (Arg) amino acid or a derivative thereof. L-Arg, D-Arg or a mixture thereof can be used according to the present invention.

Any salt or free acid of L-Arg or D-Arg, or of a derivative thereof, or mixture thereof, can be used according to the present invention. This includes but is not limited to a compound selected from the group consisting of: Arg-hydrochloride, Arg-sulfate, Arg-phosphate, Arg-citrate, Arg-acetate and Arg free acid.

An Arg derivative according to the present invention, includes but is not limited to methylated arginine, substituted L-arginine, nitro-arginine, N-nitro-L-arginine methyl ester (L-NAME), N-amino-L-arginine, N-methyl-L-arginine, monomethyl-L-arginine (L-NMA), nitro-L-arginine (L-NNA), aminoguanidine, 7-nitroindazole, S-ethylisothiourea, S-methylisothiourea, S-methylthiocitriulline, S-ethylthiocitrulline, N-ethylimino-L-ornithine, ornithine and ornithine derivatives; L-canavanine; citrulline; L-2-amino-3-guanidinopropionic acid, 4-Guanidinobutyric acid; α-N-substituted L-arginine; 2-L-arginyl-1,3-benzothiazole-6-carboxylic acid; Nω-(ADP-D-ribosyl)-L-arginine; Nω-phospho-L-arginine; N2-(2-carboxyethyl)-L-arginine; N2-succinyl-L-arginine; D-nopaline; D-octopine; L-arginine amide; L-arginine hydroxamate; argpyrimidine; hydroxy-L-arginine; methyl-L-arginine; N-acyl-L-arginine; N-benzoyl-D-arginine; Nγ-Nitro-L-arginine; Peptidyl-L-arginine.

According to some embodiments, a low-cost arginine or arginine derivative is used for the compositions of the present invention, in order to achieve relatively low-priced final product.

Any vaccine against influenza can be used in conjunction with the multimeric polypeptides in methods and compositions according to the present invention. The term “vaccines against influenza” includes but is not limited to, partially or highly purified or recombinant influenza proteins, inactivated viruses or “split product” inactivated influenza vaccine products, live attenuated viruses, or particles or carriers displaying influenza epitopes, including but not limited to virus like particles (VLP) and liposomes. Influenza vaccine to be used in conjunction with the multimeric polypeptides can be seasonal, pandemic or universal vaccines.

A conventional seasonal vaccine typically contains three inactivated or live attenuated influenza virus strains and is therefore denoted also TIV (trivalent influenza vaccine). The three strains are selected each year by the WHO to provide protection against the strains that are expected to infect in the coming season.

A non-limitative list of specific seasonal vaccines that can be used according to the present invention includes: VAXIGRIP™, AGGRIPAL™, FLUVIRIN™, FLUAD™, MUTAGRIP™, FLUZONE™, FLUZONE HD™, INFLUVAC™, FLUARIX™, FLULAVAL™, FLUMIST™, AFLURIA™, AGRIFLU™.

A pandemic vaccine typically includes one influenza virus strain specific to the relevant strain causing the pandemic. For example, The A/H1N1 strain used for swine flu pandemic during 2009/2010 season, was then included in the later seasonal TIV formulation such as the 2010/2011 season.

According to other embodiments the pandemic vaccine is against human, swine or avian influenza strains. A non-limitative list of specific pandemic vaccines that can be used according to the present invention includes: PANENZA™, PANDEMRIX™, HUMENZA™, FOCETRIA™, CELVAPAN, CELTURA™, and FLUMIST™.

### Recombinant Polypeptides

The multimeric-multiepitope polypeptides of the present invention can be prepared by expression in an expression vector per se or as a chimeric protein. The methods to produce a recombinant or chimeric protein (or polypeptide, which is interchangeably used herein), comprising one or more influenza peptide epitopes are known to those with skill in the art and are detailed, for example in WO 2009/016639. A nucleic acid sequence encoding one or more influenza peptide epitopes can be inserted into an expression vector for preparation of a polynucleotide construct for propagation and expression in host cells. A nucleic acid construct encoding a polypeptide comprising multiple repeats of several epitopes, such as a multimeric multiepitope polypeptide, can be prepared by ligation of smaller polynucleotide constructs bearing appropriated restriction sites at their 3′ and 5′ ends.

### Production of the Multimeric Polypeptide

Once expressed by the host cell, the multimeric polypeptide can be separated from undesired components by a number of protein purification methods. One such method is through the production of inclusion bodies, which are inactive aggregates of protein that may form when a recombinant polypeptide is expressed in a prokaryote. While the cDNA may properly code for a translatable mRNA, the protein that results may not fold correctly, or the hydrophobicity of the added peptide epitopes may cause the recombinant polypeptide to become insoluble. Inclusion bodies are easily purified by methods well known in the art. Various procedures for the purification of inclusion bodies are known in the art. In some embodiments the inclusion bodies are recovered from bacterial lysates by centrifugation and are washed with detergents and chelating agents to remove as much bacterial protein as possible from the aggregated recombinant protein. To obtain soluble protein, the washed inclusion bodies are typically dissolved in denaturing agents and the released protein is then refolded by gradual removal of the denaturing reagents by dilution or dialysis (for example, Molecular cloning: a laboratory manual, 3rd edition, Sambrook, J. and Russell, D. W., 2001; CSHL Press).

Another optional method uses a polyhistidine tag on the recombinant protein. A polyhistidine-tag consists in at least six histidine (His) residues added to a recombinant protein, often at the N- or C-terminus. Polyhistidine-tags are often used for affinity purification of polyhistidine-tagged recombinant proteins that are expressed in E. coli or other prokaryotic expression systems. The bacterial cells are harvested by centrifugation and the resulting cell pellet can be lysed by physical means or with detergents or enzymes such as lysozyme. The raw lysate contains at this stage the recombinant protein among several other proteins derived from the bacteria and are incubated with affinity media such as NTA-agarose, HisPur resin or Talon resin. These affinity media contain bound metal ions, either nickel or cobalt to which the polyhistidine-tag binds with micromolar affinity. The resin is then washed with phosphate buffer to remove proteins that do not specifically interact with the cobalt or nickel ion. The washing efficiency can be improved by the addition of 20 mM imidazole and proteins are then usually eluted with 150-300 mM imidazole. The polyhistidine tag may be subsequently removed using restriction enzymes, endoproteases or exoproteases. Kits for the purification of histidine-tagged proteins can be purchased for example from Qiagen.

### Vaccine Formulation and Administration

The vaccines of the present invention comprise a multiepitope polypeptide, and optionally, an adjuvant. The vaccine can be formulated for administration in one of many different modes. In one embodiment, the vaccine is formulated for parenteral administration. In some embodiments the vaccine is formulated for mass inoculation, for example for use with a jet-injector or a single use cartridge. According to one embodiment of the invention, the vaccine administration is intramuscular. According to another embodiment the administration is intradermal. Needles specifically designed to deposit the vaccine intradermally are known in the art as disclosed for example in U.S. Pat. Nos. 6,843,781 and 7,250,036 among others. According to other embodiments the administration is performed with a needleless injector.

According to yet another embodiment the vaccine is formulated for mucosal delivery, in particular nasal delivery (Amon et al., Biologicals. 2001; 29(3-4):237-42; Ben-Yedidia et al., Int Immunol. 1999; 11(7):1043-51). The vaccine formulation may be applied to the lymphatic tissue of the nose in any convenient manner. However, it is preferred to apply it as a liquid stream or liquid droplets to the walls of the nasal passage. The intranasal composition can be formulated, for example, in liquid form as nose drops, spray, and aerosol or suitable for inhalation, as powder, as cream, or as emulsion and optionally provided in a vessel appropriate for distributed the polypeptide. The composition can contain a variety of additives, such as adjuvant, excipient, stabilizers, buffers, or preservatives.

The formulations of the present invention may optionally comprise a mucosal delivery-enhancing agent such as for example a permeabilizing peptide that reversibly enhances mucosal epithelial paracellular transport by modulating epithelial junctional structure and/or physiology, as described in US 2004/0077540.

In another embodiment of the invention, administration is oral and the vaccine may be presented, for example, in the form of a tablet or encased in a gelatin capsule or a microcapsule. The formulation of these modalities is general knowledge to those with skill in the art.

Liposomes provide another delivery system for antigen delivery and presentation. Liposomes are bilayered vesicles composed of phospholipids and other sterols surrounding a typically aqueous center where antigens or other products can be encapsulated. The liposome structure is highly versatile with many types range in nanometer to micrometer sizes, from about 25 nm to about 500 μm. Liposomes have been found to be effective in delivering therapeutic agents to dermal and mucosal surfaces. The average survival time or half life of the intact liposome structure can be extended with the inclusion of certain polymers, for example polyethylene glycol, allowing for prolonged release in vivo. Liposomes may be unilamellar or multilamellar.

Polymer microparticles and nanoparticles employ small biodegradable spheres which act as depots for vaccine delivery. The major advantage that polymer microspheres possess over other depot-effecting adjuvants is that they are extremely safe and have been approved for use as a biodegradable drug delivery system. The rates of copolymer hydrolysis are very well characterized, which in turn allows for the manufacture of microparticles with sustained antigen release over prolonged periods of time (O'Hagen, et al., Vaccine. 1993, 11, 965). Parenteral administration of microparticles elicits long-lasting immunity, especially if they incorporate prolonged release characteristics. The rate of release can be modulated by the mixture of polymers and their relative molecular weights, which will hydrolyze over varying periods of time. Without wishing to be bound to theory, the formulation of different sized particles (1 μm to 200 μm) may also contribute to long-lasting immunological responses since large particles must be broken down into smaller particles before being available for macrophage uptake. In this manner a single-injection vaccine could be developed by integrating various particle sizes, thereby prolonging antigen presentation and greatly benefiting livestock producers. In some applications an adjuvant or excipient may be included in the vaccine formulation. The choice of the adjuvant is determined in part by the mode of administration of the vaccine. For example, non-injected vaccination will lead to better overall compliance and lower overall costs. One mode of administration route is intramuscular administration. Non-limiting examples of intranasal adjuvants include chitosan powder, PLA and PLG microspheres, QS-21, calcium phosphate nanoparticles (CAP) and mCTA/LTB (mutant cholera toxin E112K with pentameric B subunit of heat labile enterotoxin).

The adjuvant used may also be, theoretically, any of the adjuvants known for peptide- or protein-based vaccines. For example: inorganic adjuvants in gel form (aluminium hydroxide/aluminium phosphate, calcium phosphate, bacterial adjuvants such as monophosphoryl lipid A and muramyl peptides, particulate adjuvants such as the so-called ISCOMS (“immunostimulatory complexes”, liposomes and biodegradable microspheres, adjuvants based on oil emulsions and emulsifiers such as IFA (“Incomplete Freund's adjuvant”), SAF, saponines (such as QS-21), squalene/squalane, synthetic adjuvants such as non-ionic block copolymers, muramyl peptide analogs, synthetic lipid A, synthetic polynucleotides and polycationic adjuvants.

Another adjuvant for use with an immunogen of the present invention is an emulsion. A contemplated emulsion can be an oil-in-water emulsion or a water-in-oil emulsion. In addition to the immunogenic polypeptide, such emulsions comprise an oil phase of squalene, squalane, peanut oil or the like as are well known, and a dispersing agent. Non-ionic dispersing agents are preferred and such materials include, for example, mono- and di-C₁₂-C₂₄-fatty acid esters of sorbitan and mannide such as sorbitan mono-stearate, sorbitan mono-oleate and mannide mono-oleate.

Such emulsions are for example water-in-oil emulsions that comprise squalene, glycerol and a surfactant such as mannide mono-oleate (Arlacel™ A), optionally with squalane, emulsified with the chimer protein particles in an aqueous phase. Alternative components of the oil-phase include alpha-tocopherol, mixed-chain di- and tri-glycerides, and sorbitan esters. Well-known examples of such emulsions include Montanide™ ISA-720, and Montanide™ ISA 703 (Seppic, Castres, France). Oil-in-water emulsion adjuvants include, for example, those disclosed in WO 95/17210 and EP 0 399 843.

The use of small molecule adjuvants is also contemplated herein. One type of small molecule adjuvant useful herein is a 7-substituted-8-oxo- or 8-sulfo-guanosine derivative described in U.S. Pat. Nos. 4,539,205, 4,643,992, 5,011,828 and U.S. Pat. No. 5,093,318. 7-allyl-8-oxoguanosine(loxoribine) has been shown to be particularly effective in inducing an antigen-(immunogen-) specific response.

A useful adjuvant includes monophosphoryl lipid A (MPL®), 3-deacyl monophosphoryl lipid A (3D-MPL®), manufactured by Corixa Corp. The adjuvant contains three components extracted from bacteria: monophosphoryl lipid (MPL) A, trehalose dimycolate (TDM) and cell wall skeleton (CWS) (MPL+TDM+CWS) in a 2% squalene/Tween™ 80 emulsion. This adjuvant can be prepared by the methods taught in GB 2122204B.

Other compounds are structurally related to MPL® adjuvant called aminoalkyl glucosamide phosphates (AGPs) such as those available from Corixa Corp under the designation RC-529™ adjuvant {2-[(R)-3-tetra-decanoyloxytetradecanoylamino]-ethyl-2-deoxy-4-O-phosphon-o-3-O—[(R)-3-tetradecanoyloxytetra-decanoyl]-2-[(R)-3-tetra-decanoyloxytet-radecanoyl-amino]-p-D-glucopyranoside triethylammonium salt}. An RC-529 adjuvant is available in a squalene emulsion sold as RC-529SE and in an aqueous formulation as RC-529AF available from Corixa Corp. (disclosed for example in U.S. Pat. Nos. 6,355,257; 6,303,347; and 6,113,918).

Muramyl dipeptide adjuvants are also contemplated and include N-acetyl-muramyl-L-threonyl-D-isoglutamine (thur-MDP), N-acetyl-nor-muramyl-L-alanyl-D-isoglutamine [CGP 11637, referred to as nor-MDP], and N-acetylmuramyl-L-alanyl-D-isoglutaminyl-L-alanine-2-(1′-2′-dipalmityol-s-n-glycero-3-hydroxyphosphoryloxy) ethylamine [(CGP) 1983A, referred to as MTP-PE]. The so-called muramyl dipeptide analogues are described in U.S. Pat. No. 4,767,842.

The use of an adjuvant that contains one or more agonists for toll-like receptor-4 (TLR-4) such as an MPL® adjuvant or a structurally related compound such as an RC-529® adjuvant or a Lipid A mimetic, alone or along with an agonist for TLR-9 such as a non-methylated oligo deoxynucleotide-containing the CpG motif is also optional.

Further contemplated adjuvants include synthetic oligonucleotide adjuvants containing the CpG nucleotide motif one or more times (plus flanking sequences) available from Coley Pharmaceutical Group. The adjuvant designated QS21, available from Aquila Biopharmaceuticals, Inc., is an immunologically active saponin fractions having adjuvant activity derived from the bark of the South American tree Quillaja Saponaria Molina (e.g. Quil™ A), and the method of its production is disclosed in U.S. Pat. No. 5,057,540. Derivatives of Quil A, for example QS21 (an HPLC purified fraction derivative of Quil™ A also known as QA21), and other fractions such as QA17 are also disclosed. Semi-synthetic and synthetic derivatives of Quillaja Saponaria Molina saponins are also useful, such as those described in U.S. Pat. Nos. 5,977,081 and 6,080,725. The adjuvant denominated MF59 available from Chiron Corp. is described in U.S. Pat. Nos. 5,709,879 and 6,086,901.

Another type of adjuvant mixture comprises a stable water-in-oil emulsion further containing aminoalkyl glucosamine phosphates such as described in U.S. Pat. No. 6,113,918. Another water-in-oil emulsion is described in WO 99/56776.

Adjuvants are utilized in an adjuvant amount, which can vary with the adjuvant, host animal and immunogen. Typical amounts can vary from about 1 microgram to about 1 mg per immunization. Those skilled in the art know that appropriate concentrations or amounts can be readily determined.

According to some embodiments of the present invention, an adjuvant is present as a solution or emulsion which contains one or more water soluble or water-emulsifiable substances which are capable of making the vaccine isotonic or hypotonic. The water soluble or water-emulsifiable substances may be, for example, selected from the group consisting of: maltose; fructose; galactose; saccharose; sugar alcohol; lipid; and combinations thereof.

### Peptide Epitopes and Analogs

The multimeric polypeptides of the present invention may be synthesized chemically using methods known in the art for synthesis of peptides, peptide multimers and polypeptides. These methods generally rely on the known principles of peptide synthesis; most conveniently, the procedures can be performed according to the known principles of solid phase peptide synthesis.

As used herein “peptide” indicates a sequence of amino acids linked by peptide bonds. The peptide epitopes according to specific embodiments of the present invention comprise a sequence of 4 to 24 amino acid residues. Multimeric polypeptides comprise at least two repeats and maximum 50 repeats of the peptide epitopes.

Peptide analogs and peptidomimetics are also included within the scope of the invention when chemical synthesis is utilized. A peptide analog according to the present invention may optionally comprise at least one non-natural amino acid and/or at least one blocking group at either the C terminus or N terminus.

The term “amino acid” refers to compounds, which have an amino group and a carboxylic acid group, preferably in a 1,2- 1,3-, or 1,4-substitution pattern on a carbon backbone. α-Amino acids are most preferred, and include the 20 natural amino acids (which are L-amino acids except for glycine) which are found in proteins. When synthesis is by chemical means the term amino acid includes also the corresponding D-amino acids of the 20 natural ones, the corresponding N-methyl amino acids, side chain modified amino acids, the biosynthetically available amino acids which are not found in proteins (e.g., 4-hydroxy-proline, 5-hydroxy-lysine, citrulline, ornithine, canavanine, djenkolic acid, β-cyanolanine), and synthetically derived α-amino acids, such as amino-isobutyric acid, norleucine, norvaline, homocysteine and homoserine. β-Alanine and γ-amino butyric acid are examples of 1,3 and 1,4-amino acids, respectively, and many others are well known to the art. Statine-like isosteres (a dipeptide comprising two amino acids wherein the CONH linkage is replaced by a CHOH), hydroxyethylene isosteres (a dipeptide comprising two amino acids wherein the CONH linkage is replaced by a CHOHCH₂), reduced amide isosteres (a dipeptide comprising two amino acids wherein the CONH linkage is replaced by a CH₂NH linkage) and thioamide isosteres (a dipeptide comprising two amino acids wherein the CONH linkage is replaced by a CSNH linkage) are also useful residues for this invention.

For chemical synthesis, the amino acids used in this invention are those, which are available commercially or are available by routine synthetic methods. Certain residues may require special methods for incorporation into the peptide, and sequential, divergent or convergent synthetic approaches to the peptide sequence are useful in this invention. Natural coded amino acids and their derivatives are represented by three-letter codes according to IUPAC conventions. When there is no indication, the L isomer was used.

Conservative substitutions of amino acids as known to those skilled in the art are within the scope of the peptide epitopes of the present invention. Conservative amino acid substitutions includes replacement of one amino acid with another having the same type of functional group or side chain e.g. aliphatic, aromatic, positively charged, negatively charged. These substitutions may enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials and Methods

### Multimeric Multiepitope Polypeptides:

Multimeric-multiepitope polypeptides comprising several repeats of the influenza virus peptide epitopes E1 to E9 listed in Table 2 were produced and tested. In addition to peptide epitopes, the polypeptides include amino acids and short peptides as spacers. The polypeptides are arranged in an alternating sequential polymeric structure or a block copolymer structure. The polypeptides are prepared by expression in an expression vector from a polynucleotide construct comprising various restriction sites for further manipulation of the polypeptide. The polynucleotide construct is supplied from a commercial source.

Vaccines prepared from the multimeric-multiepitope polypeptides are used for immunization studies of various mouse models and in clinical trials in human subjects.

A multimeric polypeptide with three repeats of each of nine different epitopes arranged in block copolymer structure [E1]₃-[E2]₃-[E3]₃-[E4]₃-[E5]₃-[E6]₃-[E7]₃-[E8]₃-[E9]₃, was produced and denoted M-001. The estimated molecular weight of M-001 is 48 kD. The amino acid sequence of the M-001 multimeric polypeptide is set forth in SEQ ID NO: 86.

The DNA sequence of the polynucleotide construct used to prepare the M-001 multimeric peptide is set forth in SEQ ID NO: 85.

Additional multimeric-multiepitope polypeptide sequences are selected from the group consisting of: SEQ ID NO:84 and SEQ ID NO:88 encoded by polynucleotide sequences comprising a sequence selected from the group consisting of: SEQ ID NO:83 and SEQ ID NO:87 respectively.

Vaccine compositions according to the present invention may be administered without the need of an adjuvant. Alternatively, the composition may comprise an adjuvant. In such cases, optional adjuvant used in clinical trials is IFA in animal studies and Montanide™ ISA 51 VG (Seppic, France) in human trials. Montanide™ is a commonly used immune modulator that has been employed in many clinical trials testing vaccine efficacy, capable of inducing both cellular and humoral immune responses (Peek et al., Adv Drug Deliv Rev. 2008; 60, 915-928).

### Example 1: Production Process of M-001—Option A

### Fermentation Process:

The fermentation process was performed using heat-shock inducible E. coli recombinantly expressing the M-001 polypeptide. An overnight inoculum was added to 10 L bacterial growth medium. The fed-batch process was carried out at 36° C. Induction was initiated by increasing the temperature to 42° C. Two hours after induction, the batch was terminated at final volume of 15 L. The cells were then centrifuged and the recovered cell pellet stored at −70° C.

### Cell Lysis, Inclusion Body Recovery and Washing:

The frozen cell pellet was thawed and dispersed in Lysis Buffer (50 mM Tris, 10 mM EDTA, lysozyme). After mixing and Ultra Turrax processing, the cells were passed through a Gaulin homogenizer (700-800 bars×3 cycles). The lysate was diluted with 50 mM Tris, 0.1 M NaCl, 2% Triton X-100 and then diluted with 0.5 M Tris, 1 M NaCl. Finally the crude inclusion bodies (IBs) were recovered by centrifugation and then stored at −70° C.

The IBs pellet was then serially washed in 1% Triton; 50 mM HEPES, 1 M NaCl, pH 8; 5 M urea, 5 mM glycine, 1% Tween 80, pH 8 and finally with 50 mM HEPES, pH 8 and then stored at −70° C.

### Inclusion Bodies Solubilization:

The washed inclusion bodies were solubilized in a buffer containing 6 M urea, 2 M thiourea, 50 mM glycine, 1% CHAPS, 50 mM β-mercaptoethanol, pH 9.5.

### Protein Purification:

Purification was carried out using an AKTApilot chromatography system (GE Healthcare). Solubilized protein was clarified by membrane filtration and first loaded on a cation exchange SP Sepharose FF column (GE Healthcare). The column was washed serially with Wash Buffer I (8 M urea, 5 mM glycine, 50 mM HEPES, 50 mM β-mercaptoethanol, pH 8), then with Wash Buffer II (8 M urea, 5 mM glycine, 50 mM HEPES, 50 mM mercaptoethanol, 200 mM NaCl, pH 8) and then eluted with 8 M urea, 5 mM glycine, 50 mM HEPES, 50 mM β-mercaptoethanol, 250 mM NaCl, pH 8. The elution peak detected at 280 nm absorption, was collected.

The elute was then diluted with 1 M ammonium sulfate to 0.5 M ammonium sulfate, then loaded onto a second chromatography column, an hydrophobic interaction Phenyl Sepharose FF (HS) resin (GE Healthcare). The column was washed with 8 M urea, 5 mM glycine, 50 mM HEPES, 50 mM β-mercaptoethanol, 0.5 M ammonium sulfate, pH 8 and then eluted with 8 M urea, 5 mM glycine, 50 mM HEPES, 50 mM β-mercaptoethanol, pH 8. The elute was then passed through a Sartobind STIC (Sartorius) filter for the reduction of DNA and endotoxins. Tween-80 was added at a ratio of 1:5 (w/w) Tween 80: recombinant protein. The protein solution was aseptically filtered (0.2 μm). All subsequent steps were handled aseptically.

### Ultrafiltration and Formulation:

The elute was concentrated using a hollow fiber module with a 10 kDa cut off (GE Healthcare). In order to form protein microparticles having controlled size distribution, the buffer was serially exchanged to 50 mM MES buffer, pH 5.5, then to 50 mM citrate buffer, 0.5 M arginine, pH 5.5 and finally to 20 mM citrate buffer, 0.2 M arginine, pH 6 which represents the Drug Substance. The pharmaceutical composition—the Drug Product, is obtained by diluting the drug substance to a protein concentration of 2.5 mg/mL in 20 mM Citrate Buffer, 0.2 M arginine, pH 6. The filled vials are stored at +4° C.

### Example 2: Production Process of M-001—Option B

### Fermentation Process:

Same as in Example 1.

### Cell Lysis, Inclusion Body Recovery and Washing:

Same as in Example 1.

### Inclusion Bodies Solubilization:

The washed inclusion bodies were solubilized in a buffer containing 6 M urea, 2 M thiourea, 5 mM glycine, 1% CHAPS, 50 mM HEPES, 50 mM β-mercaptoethanol, pH 8.0.

### Protein Purification:

Purification was carried out using an AKTApilot chromatography system (GE Healthcare). Solubilized protein was clarified by membrane filtration and loaded on a cation exchange SP Sepharose FF column (GE Healthcare). The column was washed serially with Wash Buffer I (8 M urea, 5 mM glycine, 50 mM HEPES, 50 mM β-mercaptoethanol, pH 8), then with Wash Buffer II (8 M urea, 5 mM glycine, 50 mM HEPES, 50 mM β-mercaptoethanol, 200 mM NaCl, pH 8) and then eluted with 8 M urea, 5 mM glycine, 50 mM HEPES, 50 mM β-mercaptoethanol, 250 mM NaCl, pH 8. The elution peak detected at 280 nm absorption was collected.

Tween-80 was added at a ratio of 1:5 (w/w) Tween 80: recombinant protein. The protein solution was aseptically filtered (0.2 μm). All subsequent steps were handled aseptically.

### Ultrafiltration and Formulation:

The elute was concentrated using a hollow fiber module with a 10 kDa cut off (GE Healthcare). In order to form controlled microparticles (i.e., controlled protein aggregates), the buffer was serially exchanged to 50 mM MES buffer, pH 5.5, then to 50 mM citrate buffer, pH 5.5, which represents the Drug Substance. Arginine solution was added to a final arginine concentration of 0.5 M. The pharmaceutical composition—the Drug Product, was obtained by diluting the drug substance to a protein concentration of 2.5 mg/mL in 50 mM citrate buffer, 0.5 M arginine, pH 5.5. The filled vials were stored at +4° C.

### Example 3: Production Process of M-001 Testing PBS as the Drug Substance Buffer

### Fermentation Process:

Same as in Example 1.

### Cell Lysis, Inclusion Body Recovery and Washing:

Same as in Example 1.

### Inclusion Bodies Solubilization:

Same as in Example 2.

### Protein Purification:

Same as in Example 2.

### Ultrafiltration and Formulation:

The elute was diluted 1:40 with 50 mM MES buffer, pH 5.5. The resulting solution was concentrated using a hollow fiber module with a 10 kDa cut off (GE Healthcare). In order to create protein microparticles, the buffer was exchanged to PBS buffer, pH 7.0. The appearance of the protein suspension was inferior compared to the suspension obtained in Examples 1 & 2, as visually assessed by increased flocculation and precipitation. Arginine HCl was added to final arginine concentration of 0.1-1.5 M. Arginine addition caused an immediate particle deflocculation in a dose dependent manner, higher arginine concentration created more pronounced effect.

### Example 4: Production of a Freeze Dried Composition

A suspension of microparticles comprising the multimeric-multiepitope polypeptide M-001 was prepared as detailed above and kept frozen at −20° C. for 24 h. Then, the frozen composition underwent an additional freezing at −40° C. for 24 h. The frozen composition was then incubated for 48 hours at −35° C. in vacuum, using Heto power dry FD8.0 lyophilizer. The lyophilized composition was then stored at room temperature. Lyophilized M-001 compositions were found to be stable for at least 16 days at room temperature.

The lyophilized composition was then re-suspended in water and found, by visual inspection, to contain non-soluble aggregates. Since the M-001 activity does not depend on its three-dimensional structure, rather on the linear peptide sequences within it, the humoral immunity achieved by these peptide epitopes is maintained.

1. A pharmaceutical composition in the form of an aqueous suspension of microparticles, said composition comprising at least one multimeric-multiepitope polypeptide comprising multiple copies of plurality of influenza virus peptide epitopes, a guanidinium-containing amino acid or a derivative thereof, and a pharmaceutically-acceptable diluent, excipient or carrier, wherein the aqueous suspension comprises aggregates of microparticles of said multimeric-multiepitope polypeptide having uniform aggregate size distribution with 95% of the aggregates in the suspension having a size range distribution selected from the group consisting of: 0.5-5 μm, 0.6-6 μm, 0.7-7 μm, 0.8-8 μm, 0.9-9 μm, 1-10 μm, 2-20 μm, 3-30 μm, 4-40 μm and 5-50 μm.

2. The pharmaceutical composition according to claim 1, wherein the guanidinium-containing amino acid is an arginine (Arg).

3. The pharmaceutical composition according to claim 1 wherein the composition comprises a buffering agent at a concentration of 1-50 mM that maintains a pH within the range of 5.0 to 7.6.

4. The pharmaceutical composition according to claim 1, comprising 1-10 mg/ml of multimeric-multiepitope influenza polypeptide, 0.1-0.5 M of L-arginine and 10-50 mM citrate buffer, and having a pH in the range of 4 to 7.

5. The pharmaceutical composition according to claim 1, wherein the multimeric-multiepitope polypeptide comprises 3-5 repeats of 4-9 peptide epitopes each epitope is selected from the group consisting of: HA 354-372 (E1, SEQ ID NO: 82), HA 91-108 (E2, SEQ ID NO: 48), M1 2-12 (E3, SEQ ID NO: 25), HA 150-159 (E4, SEQ ID NO: 52), HA 143-149 (E5, SEQ ID NO: 51), NP 206-229 (E6, SEQ ID NO: 64), HA 307-319 (E7, SEQ ID NO: 59 or 89), NP 335-350 (E8, SEQ ID NO: 69), and NP 380-393 (E9, SEQ ID NO: 70).

6. The pharmaceutical composition according to claim 5, wherein the multimeric-multiepitope influenza polypeptide is M-001 having an amino acid sequence set forth in SEQ ID NO: 86.

7. The pharmaceutical composition according to claim 1, in a form suitable for administration in a route selected from injectable, oral, intranasal and sublingual.

8. The pharmaceutical composition according to claim 1, wherein the aqueous suspension of microparticles is produced by a process comprising the steps of:
i. providing at least one multimeric multiepitope influenza polypeptide at a concentration of 1.5-5 mg/ml in an aqueous solution comprising a chaotropic agent, a detergent, a reducing agent, and a buffering agent providing a pH in the range of 7-10;
ii. inducing of controlled aggregation by gradual removal of the chaotropic and reducing agents; and
iii. adding a guanidinium-containing compound to achieve a stable microparticulate suspension having a uniform aggregate size distribution, with 95% of the aggregate sizes falling into a size range of one order of magnitude.

9. A process of producing a recombinant multimeric-multiepitope polypeptide composition, comprising the steps:
i. providing at least one multimeric multiepitope influenza polypeptide in an aqueous solution comprising a chaotropic agent, a detergent, a reducing agent, and a buffering agent, providing a pH in the range of 7-11;
ii. inducing of controlled aggregation by gradual removal of the chaotropic and reducing agents; and
iii. adding a guanidinium-containing amino acid or a derivative thereof to achieve a stable microparticulate suspension having a uniform aggregate size distribution, with 95% of the aggregate in the suspension having a size range distribution selected from the group consisting of: 0.5-5 μm, 0.6-6 μm, 0.7-7 μm, 0.8-8 μm, 0.9-9 μm, 1-10 μm, 2-20 μm, 3-30 μm, 4-40 μm and 5-50 μm;
wherein the at least one multimeric-multiepitope polypeptide comprises multiple copies of plurality of influenza virus peptide epitopes.

10. The process according to claim 9, further comprising the steps of:
iv. freezing the stable microparticulate suspension of step (iii) at a temperature in the range of −10° C. to −50° C.; and
v. reducing the pressure of the frozen composition of step (iv) for a period of time, so as to sublime at least 95% of the water contained therein, thereby providing a solid composition of the multimeric multiepitope influenza polypeptide.

11. The process according to claim 10, wherein step (iv) comprises freezing the stable microparticulate suspension of step (iii) at a temperature in the range of −15° C. to −25° C. for 12 hours to 36 hours, and reducing said temperature to a temperature in the range of −30° C. to −50° C. for further 12 hours to 36 hours.

12. The process according to claim 11, wherein step (v) comprises reducing the pressure of the frozen composition of step (iv) for a period of 24 hours to 72 hours, so as to sublime at least 98% of the water contained therein, thereby providing the solid composition of the multimeric multiepitope influenza polypeptide.

13. The process according to claim 9, wherein step (i) comprises providing at least one multimeric multiepitope influenza polypeptide at a concentration of 1.5-5 mg/ml in an aqueous solution comprising a chaotropic agent, a detergent, a reducing agent, and a buffering agent providing a pH in the range of 7-10.

14. The process according to claim 9, wherein the chaotropic agent is 5-8 M urea and 1-4 M thiourea, the detergent is 0.5-4% CHAPS (3-[(3-cholamidopropyl) dimethylammonio]-1-propanesulfonate) and the buffering agent is 5-100 mM glycine, and wherein the guanidinium-containing amino acid is an arginine (Arg).

15. A pharmaceutical composition of multimeric multiepitope influenza polypeptide, comprising a stable microparticulate suspension prepared according to the process of claim 9, and a pharmaceutically-acceptable diluent, excipient or carrier.

16. A solid pharmaceutical composition comprising a solid composition of a multimeric multiepitope influenza polypeptide prepared according to the process of claim 10, and a pharmaceutically-acceptable excipient or carrier.

17. The pharmaceutical composition according to claim 16, in the form of a powder.

18. A packaged pharmaceutical composition comprised of a packaging material; and the solid pharmaceutical composition of claim 16.

19. A method of inducing an immune response and conferring protection against influenza in a subject, comprising administering to the subject a pharmaceutical composition according to claim 16.

20. The method according to claim 19, wherein the administration is intranasal.
